SEQUENCE LISTING (12 sequences)

SEQ ID NO: 1 - protein, 602 aa, Tribolium castaneum
MTMDRVEKNVKKRKYSEDFLQYGFTSIITAGIEKPQCVICCEVLSAESMKPNKLKRHFDSKHPSFAGKDTNYFRSKADGLKKARLDTGGKYHKQNVAAIEASYLVALRIARAMKPHTIAEDLLLPAAKDIVRVMIGDEFVTKLSAISLSNDTVRRRIDDMSADILDQVIQEIKSAPLPIFSIQLDESTDVANCSQLLVYVRYINDGDFKDEFLFCKPLEMTTTARDVFDTVGSFLKEHKISWEKVCGVCTDGAPAMLGCRSGFQRLVLNESPKVIGTHCMIHRQILATKTLPQELQEVMKSVISSVNFVKASTLNSRLFSQLCNELDAPNNALLFHTEVRWLSRGKVLKRVFELRDELKTFFNQKARPQFEALFSDKSELQKIAYLVDIFAILNELNLSLQGPNATCLDLSEKIRSFQMKLQLWQKKLDENKIYMLPTLSAFFEEHDIEPDKRITMIISVKEHLHMLADEISSYFPNLPDTPFALARSPFTVKVEDVPETAQEEFIELINSDAARTDFSTMPVTKFWIKCLQSYPVLSETVLRLLLPFPTTYLCETGFSSLLVIKSKYRSRLVVEDDLRCALAKTAPRISDLVRKKQSQPSH

SEQ ID NO: 2 - DNA, 1809 bp, Tribolium castaneum
atgaccatggaccgcgttgaaaagaatgtaaaaaagagaaagtatagtgaggatttcttgcagtatggatttacttccattatcactgcgggtattgaaaaacctcaatgtgttatatgttgcgaagtcctgtcagcagaatcaatgaaacctaataaattgaaaaggcatttcgattccaagcatccaagcttcgctgggaaggataccaactattttaggtctaaagctgacggtcttaaaaaagcgcggttggatacaggtggtaagtaccacaagcagaatgtggcggctatcgaggcgtcctatctggttgcacttcgcatcgctagagctatgaaaccacacaccatcgcagaggatcttctccttcccgccgcaaaagatatcgttcgagtgatgataggagatgaattcgtaacgaaactttctgcgatatcactctcaaacgacacggtacgacggaggatagatgacatgagtgctgacatattggaccaggtgatacaggaaattaagtctgctccccttccgatattctctattcaactcgacgaaagcaccgatgttgcaaattgttcacagttgttggtatatgtacggtatattaatgatggggatttcaaagatgagttcctgttttgtaagcctcttgaaatgaccaccacagcccgggatgtattcgacactgtcggtagcttccttaaagaacacaaaattagctgggaaaaggtctgtggtgtttgtacggacggtgctccggcgatgctgggatgcagatcaggatttcaaagactcgtgcttaacgagtctcctaaggtgataggcactcactgtatgatacaccggcaaattctcgcaaccaagacattgccacaggaacttcaagaagttatgaagtctgtaatatcatccgtaaatttcgtgaaagcaagtactctgaactcacgactcttttcacaactttgtaatgagcttgacgcacccaacaacgccctgttgtttcatacagaagtccggtggctgagtcgcgggaaagtacttaagagggtattcgagctccgggacgagctgaagacatttttcaaccagaaggcacgaccccaatttgaggcgctgtttagcgataagagcgaacttcagaagatcgcgtaccttgtggatatcttcgcaattttgaacgaactcaacttgtctctgcaagggcctaatgccacgtgcctggacctttccgagaagattagatccttccagatgaagttgcagctgtggcagaaaaagctggatgaaaacaagatttatatgttgccgacactttccgcatttttcgaggaacacgacattgaaccagacaaacgcatcacaatgattatctcagtgaaagagcacttgcacatgttggccgacgaaatttcatcctattttccaaatcttccagatactccgttcgctctcgcacgcagccctttcacggtaaaagttgaagacgtaccagaaacggcacaggaggagttcattgaactgattaattctgatgctgcccgcactgacttttccacgatgccagttacgaaattttggattaaatgtcttcagtcctatcccgttcttagtgagacggtattgcggcttcttctcccatttccgaccacgtacctctgtgaaacgggattctcatccttgctggtgatcaaaagcaagtaccgatcccgactcgtggtcgaagatgaccttcgatgcgccctcgcaaaaactgcaccccggatcagcgacttggtgagaaagaaacaatctcaaccaagtcactga

SEQ ID NO: 3 - DNA, 703 bp, Artificial sequence
  Synthetic
cagcggttctcaacctgtgggtcgcgacccctttgggggtcaaacgaccctttcacaggggtcgcctaagaccatcggaaaacacatatttccgatggtcttaggaaccgagacaccgctcctctatccgtctccaggcgggtccgcccacatgcagatacgcccacataggagtacccggcgtgatgacatcatcgcgccaaccccatcacatacaccccgtacaaatacaggtgtatgtgacagggttggcgccataatgtacttatgcggaccagtcacacatgtgtagagagcagctactgtgtagaaagcagctactgtgttgaaagcagctactctgttaaaagcagctactgtgttgaaagcagcagtattggaggtaaaacgacacttcatgaattataattactgggtaaatgtaaaattcatgtactgtaaaatcatcaactactgcaaaaaaaaaaaaatatctgtaccatggggaacttaatctggatgctgatcggtctttttatattcagctgtggttgatgtgaatactgcccccttgtgatagtaacaggtatgtaaaaaaaaacaacacagagaatggtaaatcataggaaactttaatgaactgtattgactgaactatgccatgtatcatcttttgtattattaaagctattgttatatattattttcattagcaaaccatccc

SEQ ID NO: 4 - DNA, 361 bp, Artificial sequence
  Synthetic
cgttggctttttacgcatactgtcgcaaaatgtagcaatgtagtttactgttgttatattaagactgttacccatgctacaccatgcttcaagacaaaatttcatttatttgtaattagaaataaatatttcacaatatataattacatattgtttttgtgattaatcactatgctttaattatgttcaatttgtaacaatgaaaatacatcctgcatatcagatatttacattacgattcataacagtagcaaaattacagttatgaagtagcaacgaaaataattttatggttgggggtcaccacaacatgaggaactgtattaaagggtcgcggcattaggaaggttgagaaccactg

SEQ ID NO: 5 - DNA, 7143 bp, Artificial sequence
  Synthetic
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 2742 bp, Artificial sequence
  Synthetic
atgctcgagatggatccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgttgactgatgctggtgagcttagaggacctcctttgcaacttgatacaggccagcttctgaaaatcgccaagaggggtggggtcaccgcggtcgaggccgtacacgcctggagaaatgcactgaccggggctcctcttaacctgaccccagaccaggtagtcgcaatcgcgtcaaacggagggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgacggccaagctggccgggggcgcccccgccgtgggcgggggccccaaggccgccgataaattcgccgccaccatgaccatggaccgcgttgaaaagaatgtaaaaaagagaaagtatagtgaggatttcttgcagtatggatttacttccattatcactgcgggtattgaaaaacctcaatgtgttatatgttgcgaagtcctgtcagcagaatcaatgaaacctaataaattgaaaaggcatttcgattccaagcatccaagcttcgctgggaaggataccaactattttaggtctaaagctgacggtcttaaaaaagcgcggttggatacaggtggtaagtaccacaagcagaatgtggcggctatcgaggcgtcctatctggttgcacttcgcatcgctagagctatgaaaccacacaccatcgcagaggatcttctccttcccgccgcaaaagatatcgttcgagtgatgataggagatgaattcgtaacgaaactttctgcgatatcactctcaaacgacacggtacgacggaggatagatgacatgagtgctgacatattggaccaggtgatacaggaaattaagtctgctccccttccgatattctctattcaactcgacgaaagcaccgatgttgcaaattgttcacagttgttggtatatgtacggtatattaatgatggggatttcaaagatgagttcctgttttgtaagcctcttgaaatgaccaccacagcccgggatgtattcgacactgtcggtagcttccttaaagaacacaaaattagctgggaaaaggtctgtggtgtttgtacggacggtgctccggcgatgctgggatgcagatcaggatttcaaagactcgtgcttaacgagtctcctaaggtgataggcactcactgtatgatacaccggcaaattctcgcaaccaagacattgccacaggaacttcaagaagttatgaagtctgtaatatcatccgtaaatttcgtgaaagcaagtactctgaactcacgactcttttcacaactttgtaatgagcttgacgcacccaacaacgccctgttgtttcatacagaagtccggtggctgagtcgcgggaaagtacttaagagggtattcgagctccgggacgagctgaagacatttttcaaccagaaggcacgaccccaatttgaggcgctgtttagcgataagagcgaacttcagaagatcgcgtaccttgtggatatcttcgcaattttgaacgaactcaacttgtctctgcaagggcctaatgccacgtgcctggacctttccgagaagattagatccttccagatgaagttgcagctgtggcagaaaaagctggatgaaaacaagatttatatgttgccgacactttccgcatttttcgaggaacacgacattgaaccagacaaacgcatcacaatgattatctcagtgaaagagcacttgcacatgttggccgacgaaatttcatcctattttccaaatcttccagatactccgttcgctctcgcacgcagccctttcacggtaaaagttgaagacgtaccagaaacggcacaggaggagttcattgaactgattaattctgatgctgcccgcactgacttttccacgatgccagttacgaaattttggattaaatgtcttcagtcctatcccgttcttagtgagacggtattgcggcttcttctcccatttccgaccacgtacctctgtgaaacgggattctcatccttgctggtgatcaaaagcaagtaccgatcccgactcgtggtcgaagatgaccttcgatgcgccctcgcaaaaactgcaccccggatcagcgacttggtgagaaagaaacaatctcaaccaagtcactga

SEQ ID NO: 7 - protein, 15 aa, Artificial sequence
  Synthetic
GGSGGSGGSGGSGTS

SEQ ID NO: 8 - DNA, 45 bp, Artificial sequence
  Synthetic
ggaggtagtggcggtagtgggggctccggtgggagcggcacctca

SEQ ID NO: 9 - DNA, 864 bp, Artificial sequence
  Synthetic
atgctcgagatggatccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgttgactgatgctggtgagcttagaggacctcctttgcaacttgatacaggccagcttctgaaaatcgccaagaggggtggggtcaccgcggtcgaggccgtacacgcctggagaaatgcactgaccggggctcctcttaacctgaccccagaccaggtagtcgcaatcgcgtcaaacggagggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctccaatattggcggtaagchaggcgctggaaacagtacagcgcctgctgcctgtactgtgccaggatcatggactgac

SEQ ID NO: 10 - DNA, 70 bp, Artificial sequence
  Synthetic
ggccaagctggccgggggcgcccccgccgtgggcgggggccccaaggccgccgataaattcgccgccacc

SEQ ID NO: 11 - protein, 602 aa, Artificial sequence
  Synthetic
MTMDRVEKNVKKRKYSEDFLQYGFTSIITAGIEKPQCVICCEVLSAESMKPNKLKRHFDSKHPSFAGKDTNYFRSKADGLKKARLDTGGKYHKQNVAAIEASYLVALRIARAMKPHTIAEDLLKPAAKDIVRVMIGDEFVTKLSAISLSNDTVRRRIDDMSADILDQVIQEIKSAPLPIFSIQLDESTDVANCSQLLVYVRYINDGDFKDEFLFCKPLEMTTTARDVFDTVGSFLKEHKISWEKVCGVCTDGAPAMLGCRSGFQRLVLNESPKVIGTHCMIHRQILATKTLPQELQEVMKSVISSVNFVKASTLNSRLFSQLCNELDAPNNALLFHTEVRWLSRGKVLKRVFELRDELKTFFNQKARPQFEALFSDKSELQKIAYLVDIFAILNELNLSLQGPNATCLDLSEKIRSFQMKLQLWQKKLDENKIYMLPTLSAFFEEHDIEPDKRITMIISVKEHLHMLADEISSYFPNLPDTPFALARSPFTVKVEDVPETAQEEFIELRNSDAARTDFSTMPVTKFWIKCLQSYPVLSETVLRLLLPFPTTYLCETGFSSLLVIKSKYRSRLVVEDDLRCALAKTAPRISDLVRKKQSQPSH

SEQ ID NO: 12 - protein, 636 aa, Tribolium castaneum
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

(19) United States
(12) Patent Application Publication
(10) Pub. No.: US 2023/0272355 A1
(43) Pub. Date: Aug. 31, 2023

(54) ENHANCED hAT FAMILY MEMBER SPIN TRANSPOSON-MEDIATED GENE TRANSFER AND ASSOCIATED COMPOSITIONS, SYSTEMS, AND METHODS

(71) Applicant: B-MOGEN BIOTECHNOLOGIES, INC., Minneapolis, MN (US)

(72) Inventors: David Largaespada, Mounds View, MN (US); Branden Moriarity, Shoreview, MN (US); Beau Webber, Coon Rapids, MN (US); Neil Otto, Lester Prairie, MN (US); Sandeep Kumar, Minneapolis, MN (US); Bryan Jones, Minneapolis, MN (US)

(21) Appl. No.: 18/000,634

(22) PCT Filed: Jun. 4, 2021
(86) PCT No.: PCT/US2021/035888

§ 371 Date: Dec. 2, 2022

Related U.S. Application Data
(60) Provisional application No. 63/035,441, filed on Jun. 5, 2020.

(51) Int. Cl. C12N 9/12; C12N 9/22; C12N 15/63; C12N 15/62; C12N 5/0783; A61K 35/17; A61P 35/00

(52) U.S. Cl. CPC C12N 9/1241; C12N 9/22; C12N 15/63; C12N 15/62; C12N 5/0636; A61K 35/17; A61P 35/00; C12N 2510/00

(57) ABSTRACT

This disclosure provides various SPIN transposases and transposons, systems, and methods of use.

### CROSS-REFERENCE TO RELATED APPLICATIONS

This application claims the benefit of U.S. Provisional Application No. 63/035,441, filed Jun. 5, 2020, the contents of which are incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 35,527 Byte ASCII (Text) file named “37963-601_ST25.TXT,” created on Jun. 4, 2021.

### BACKGROUND

Transposable genetic elements, also called transposons, are segments of DNA that can be mobilized from one genomic location to another within a single cell. Transposons can be divided into two major groups according to their mechanism of transposition: transposition can occur (1) via reverse transcription of an RNA intermediate for elements termed retrotransposons, and (2) via direct transposition of DNA flanked by terminal inverted repeats (TIRs) for DNA transposons. Active transposons encode one or more proteins that are required for transposition. The natural active DNA transposons harbor a transposase enzyme gene.

DNA transposons in the hAT family are widespread in plants and animals. A number of active hAT transposon systems have been identified and found to be functional, including but not limited to, the Hermes transposon, Ac transposon, hobo transposon, and the Tol2 transposon. The hAT family is composed of two families that have been classified as the AC subfamily and the Buster subfamily, based on the primary sequence of their transposases. Members of the hAT family belong to Class II transposable elements. Class II mobile elements use a cut and paste mechanism of transposition. hAT elements share similar transposases, short terminal inverted repeats, and an eight base-pairs duplication of genomic target.

### SUMMARY

One aspect of the present disclosure provides a mutant SPIN transposase comprising an amino acid sequence at least 70% identical to full-length SEQ ID NO: 1 and having increased transposition efficiency in comparison to a wild-type SPIN transposase having amino acid sequence SEQ ID NO: 1.

Another aspect of the present disclosure provides a mutant SPIN transposase comprising an amino acid sequence at least 70% identical to full-length SEQ ID NO: 1 and having one or more amino acid substitutions that increase a net charge at a neutral pH in comparison to SEQ ID NO: 1. In some cases, the mutant SPIN transposase has increased transposition efficiency in comparison to a wild-type SPIN transposase having amino acid sequence SEQ ID NO: 1.

Another aspect of the present disclosure provides a mutant SPIN transposase comprising an amino acid sequence at least 70% identical to full-length SEQ ID NO: 1 and having one more amino acid substitutions in a Specific End Binding Domain: an insertion domain; a Zn-BED domain; or a combination thereof. In some cases, the mutant SPIN transposase has increased transposition efficiency in comparison to a wild-type SPIN transposase having amino acid sequence SEQ ID NO: 1. In some cases, the transposition efficiency is measured by an assay that comprises introducing the mutant SPIN transposase and a SPIN transposon containing a reporter cargo cassette into a population of cells and detecting transposition of the reporter cargo cassette in genome of the population of cells.

Another aspect of the present disclosure provides a mutant SPIN transposase comprising an amino acid sequence at least 70% identical to full-length SEQ ID NO: 1 and having one or more amino acid substitutions from Table 1. In some cases, a mutant SPIN transposase comprises one or more amino acid substitutions that increase a net charge at a neutral pH within or in proximity to a catalytic domain in comparison to SEQ ID NO: 1. In some cases, a mutant SPIN transposase comprises one or more amino acid substitutions that increase a net charge at a neutral pH in comparison to SEQ ID NO: 1, wherein the one or more amino acids are located in proximity to D185, D251, or E555, when numbered in accordance to SEQ ID NO: 1.

Another aspect of the present disclosure provides a fusion transposase comprising a SPIN transposase sequence and a DNA sequence specific binding domain. In some case, a SPIN transposase sequence has at least 70% identity to full-length SEQ ID NO: 1.

Another aspect of the present disclosure provides a polynucleotide that codes for a mutant SPIN transposase as described herein.

Another aspect of the present disclosure provides a polynucleotide that codes for a fusion transposase as described herein.

Another aspect of the present disclosure provides a cell producing a mutant SPIN transposase or a fusion transposase as described herein.

Another aspect of the present disclosure provides a cell containing a polynucleotide as described herein.

Another aspect of the present disclosure provides a method of genome editing, comprising: introducing into a cell a mutant SPIN transposase as described herein and a transposon recognizable by the mutant SPIN transposase.

Another aspect of the present disclosure provides a method of genome editing, comprising: introducing into a cell a fusion transposase as described herein and a transposon recognizable by the fusion transposase.

Another aspect of the present disclosure provides a method of treatment, comprising: (a) introducing into a cell a transposon and a mutant SPIN transposase or a fusion transposase as described herein, which recognize the transposon, thereby generating a genetically modified cell; (b) administering the genetically modified cell to a patient in need of the treatment.

Another aspect of the present disclosure provides a system for genome editing, comprising: a mutant SPIN transposase or fusion transposase as described herein, and a transposon recognizable by the mutant SPIN transposase or the fusion transposase.

Another aspect of the present disclosure provides a system for genome editing, comprising: a polynucleotide encoding a mutant SPIN transposase or fusion transposase as described herein, and a transposon recognizable by the mutant SPIN transposase or the fusion transposase.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent that a term incorporated by reference conflicts with a term defined herein, this specification controls.

### BRIEF DESCRIPTION OF THE DRAWINGS

FIG. 1 is a schematic of protein domains an exemplary SPIN transposase.

FIG. 2 depicts the amino acid sequence alignment of SPIN transposase versus a number of other transposase members in Buster subfamily. Exemplary potential activating substitutions are indicated above the SPIN protein sequence. Alignments were performed using T-Coffee Multiple Sequence Alignment. Different colors representing BAD, AVG, and GOOD indicate the different conservation qualities of protein sequences.

FIG. 3 shows a vector map of an exemplary expression vector pcDNA-DEST40 that was used to test SPIN transposase mutants.

FIG. 4 is a graph quantifying the transposition efficiency of exemplary SPIN transposase mutants, as measured by percent of mCherry positive cells in HEK-293T cells that were transfected with SPIN transposon Tn with the exemplary transposase mutants.

FIG. 5 shows amino acid sequence of wild-type SPIN transposase with certain amino acids annotated (SEQ ID NO: 1).

### DETAILED DESCRIPTION

### Overview

DNA transposons can translocate via a non-replicative, ‘cut-and-paste’ mechanism. This requires recognition of the two terminal inverted repeats by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two inverted repeats and may contain a gene encoding a transposase that catalyzes transposition.

For genome editing applications with DNA transposons, it is desirable to design a transposon to develop a binary system based on two distinct plasmids whereby the transposase is physically separated from the transposon DNA containing the gene of interest flanked by the inverted repeats. Co-delivery of the transposon and transposase plasmids into the target cells enables transposition via a conventional cut-and-paste mechanism.

SPIN is a member of the hAT family of DNA transposons. Other members of the family include Sleeping Beauty and PiggyBac. Discussed herein are various devices, systems and methods relating to synergistic approaches to enhance gene transfer into human hematopoictic and immune system cells using hAT family transposon components. The present disclosure relates to improved hAT transposases, transposon vector sequences, transposase delivery methods, and transposon delivery methods. In one implementation, the present study identified specific, universal sites for making hyperactive hAT transposases. In another implementation, methods for making minimally sized hAT transposon vector inverted terminal repeats (ITRs) that conserve genomic space are described. In another implementation, improved methods to deliver hAT family transposases as chemically modified in vitro transcribed mRNAs are described. In another implementation, methods to deliver hAT family transposon vectors as “miniature” circles of DNA are described, in which virtually all prokaryotic sequences have been removed by a recombination method. In another implementation, methods to fuse DNA sequence specific binding domains using transcription activator-like (TAL) domains fused to the hAT transposases are described. These improvements, individually or in combination, can yield unexpectedly high levels of gene transfer to the cell types in question and improvements in the delivery of transposon vectors to sequences of interest.

### Mutant SPIN Transposase

One aspect of the present disclosure provides a mutant SPIN transposase. A mutant SPIN transposase may comprise one or more amino acid substitutions in comparison to a wild-type SPIN transposase (SEQ ID NO: 1).

A mutant SPIN transposase can comprise an amino acid sequence having at least 70% sequence identity to full length sequence of a wild-type SPIN transposase (SEQ ID NO: 1). In some embodiments, a mutant SPIN transposase can comprise an amino acid sequence having at least 40%, at least 50%, at least 60%, at least 70%, at least 80%, at least 90%, at least 95%, at least 96%, at least 97%, at least 98%, or at least 99% sequence identity to full length sequence of a wild-type SPIN transposase (SEQ ID NO: 1). In some cases, a mutant SPIN transposase can comprise an amino acid sequence having at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9%, or at least 99.95% sequence identity to full length sequence of a wild-type SPIN transposase (SEQ ID NO: 1).

A mutant SPIN transposase can comprise an amino acid sequence having at least one amino acid different from full length sequence of a wild-type SPIN transposase (SEQ ID NO: 1). In some embodiments, a mutant SPIN transposase can comprise an amino acid sequence having at least 2, at least 3, at least 4, at least 5, at least 6, at least 7, at least 8, at least 9, at least 10, or more amino acids different from full length sequence of a wild-type SPIN transposase (SEQ ID NO: 1). In some cases, a mutant SPIN transposase can comprise an amino acid sequence having at least 5, at least 10, at least 20, at least 30, at least 40, at least 50, at least 60, at least 70, at least 80, at least 90, at least 100, at least 200, or at least 300 amino acid different from full length sequence of a wild-type SPIN transposase (SEQ ID NO: 1). In some cases, a mutant SPIN transposase can comprise an amino acid sequence having at most 3, at most 6, at most 12, at most 25, at most 35, at most 45, at most 55, at most 65, at most 75, at most 85, at most 95, at most 150, at most 250, or at most 350 amino acid different from full length sequence of a wild-type SPIN transposase (SEQ ID NO: 1).

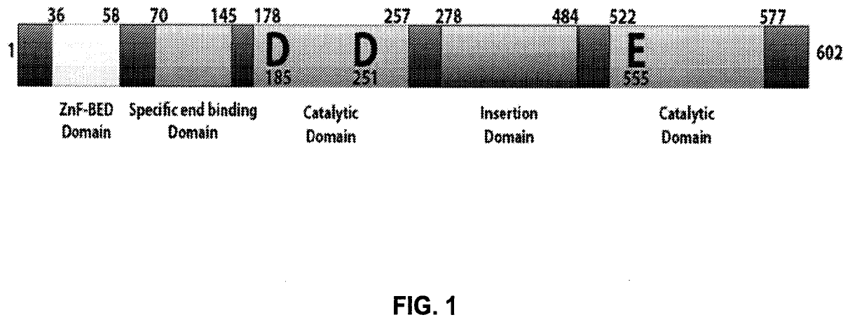

As shown in FIG. 1, typically, a wild-type SPIN transposase can be regarded as comprising, from N terminus to C terminus, a ZnF-BED domain (amino acids 36-58), a Specific End Binding Domain (amino acids 70-145), a first Catalytic domain (amino acids 178-257), an Insertion domain (amino acids 278-484), and a second Catalytic domain (amino acids 522-577), as well as at least four inter-domain regions in between these annotated domains. Unless indicated otherwise, numerical references to amino acids, as used herein, are all in accordance to SEQ ID NO: 1. A mutant SPIN transposase can comprise one or more amino acid substitutions in any one of these domains, or any combination thereof. In some cases, a mutant SPIN transposase can comprise one or more amino acid substitutions in ZnF-BED domain, a Specific End Binding Domain, a first Catalytic domain, an Insertion domain, or a combination thereof. A mutant SPIN transposase can comprise one or more amino acid substitutions in at least one of the two catalytic domains.

An exemplary mutant SPIN transposase can comprise one or more amino acid substitutions from Table 1. Sometimes, a mutant SPIN transposase can comprise at least one of the amino acid substitutions from Table 1. A mutant SPIN transposase can comprise at least 2, at least 3, at least 4, at least 5, at least 6, at least 7, at least 8, at least 9, at least 10, at least 20, at least 30, or more of the amino acid substitutions from Table 1.

TABLE 1

| Amino Acid of Wild-type | Amino Acid |
|---|---|
| SPIN Transposase | Substitution |
| I509 | I509R |
| I509 | I509S |
| P549 | P549S |
| P549 | P549A |
| P329 | P329D |
| A332 | A332D |
| A332 | A332S |
| E379 | E379W |
| A486 | A486S |
| S511 | S511N |
| M220 | M220T |
| L266 | L266R |
| I509 + I511 | I509R + I511R |
| L266 | L266K |
| E100 | E100K |
| N204 | N204K |
| L121 | L121K |
| E539 | E539R |
| E219 | E219K |
| N269 | N269K |
| E171 | E171K |
| L124 | L124K |
| N204 | N204H |
| I99 | I99V |
| I99 | I99L |
| T360 | T360M |
| T313 | T313S |
| T313 | T313A |
| L436 | L436F |
| D210 | D210E |
| N330 | N330H |
| I285 | I285A |

An exemplary mutant SPIN transposase comprises one or more amino acid substitutions, or combinations of substitutions, from Table 2. Sometimes, a mutant SPIN transposase can comprise at least one of the amino acid substitutions, or combinations of substitutions, from Table 2. A mutant SPIN transposase can comprise at least 2, at least 3, at least 4, at least 5, at least 6, at least 7, at least 8, at least 9, at least 10, at least 20, at least 30, or more of the amino acid substitutions, or combinations of substitutions, from Table 2.

TABLE 2

| Amino Acid of Wild-type | Amino Acid |
|---|---|
| SPIN Transposase | Substitution |
| I509 | I509R |
| I509 | I509S |
| S511 | S511N |
| L124 | L124K |
| E219 | E219K |
| A332 | A332D |
| Y595 | Y595L |
| T598 | T598I |
| P254 | P254A |
| A581 | A581Q |
| Q195 | Q195I |
| C41 | C41E |
| E42 | E42Q |
| A332 | A332N |
| F335 | F335L |
| V351 | V351F |

An exemplary mutant SPIN transposase comprises one or more combinations of amino acid substitutions from Table 3. Sometimes, a mutant SPIN transposase can comprise at least one of the combinations of amino acid substitutions from Table 3. A mutant SPIN transposase can comprise at least 2, at least 3, at least 4, at least 5, at least 6, at least 7, at least 8, at least 9, at least 10, at least 20, at least 30, or more of the combinations of amino acid substitutions from Table 3 with one of the respective designated substitutions.

TABLE 3

| Amino Acid of Wild-type | Amino Acid |
|---|---|
| SPIN Transposase | Substitution |
| I509 | I509R |
| I509 | I509S |
| S511 | S511N |
| L124 | L124K |
| E219 | E219K |

“Identical” and its grammatical equivalents as used herein or “sequence identity” in the context of two nucleic acid sequences or amino acid sequences of polypeptides can refer to the residues in the two sequences which are the same when aligned for maximum correspondence over a specified comparison window. A “comparison window”, as used herein, can refer to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988): by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison. Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992): and Pearson et al., Methods in Molecular Biology, 24:307-331 (1994). Alignment is also often performed by inspection and manual alignment. In one class of embodiments, the polypeptides herein have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99% or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

### Hyperactive Mutant SPIN Transposase

Another aspect of the present disclosure is to provide a hyperactive mutant SPIN transposase. A “hyperactive” mutant SPIN transposase, as used herein, can refer to any mutant SPIN transposase that has increased transposition efficiency as compared to a wild-type SPIN transposase having amino acid sequence SEQ ID NO: 1.

In some embodiments, a hyperactive mutant SPIN transposase may have increased transposition efficiency under certain situations as compared to a wild-type SPIN transposase having amino acid sequence SEQ ID NO: 1. For example, the hyperactive mutant SPIN transposase may have better transposition efficiency than the wild-type SPIN transposase when being used to catalyze transposition of transposons having particular types of inverted repeat sequences. It is possible that with some other transposons having other types of inverted repeat sequences, the hyperactive mutant SPIN transposase does not have increased transposition efficiency in comparison to the wild-type SPIN transposase. In some other non-limiting examples, the hyperactive mutant SPIN transposase may have increased transposition efficiency in comparison to a wild-type SPIN transposase having amino acid sequence SEQ ID NO: 1, under certain transfection conditions. Without being limited, when compared to a wild-type SPIN transposase, a hyperactive mutant SPIN transposase may have better transposition efficiency when the temperature is higher than normal cell culture temperature; a hyperactive mutant SPIN transposase may have better transposition efficiency in a relative acidic or basic aqueous medium; a hyperactive mutant SPIN transposase may have better transposition efficiency when a particular type of transfection technique (e.g., electroporation) is performed.

Transposition efficiency can be measured by the percent of successful transposition events occurring in a population of host cells normalized by the amount of transposon and transposase introduced into the population of host cells. In many instances, when the transposition efficiency of two or more transposases is compared, the same transposon construct is paired with each of the two or more transposases for transfection of the host cells under same or similar transfection conditions. The amount of transposition events in the host cells can be examined by various approaches. For example, the transposon construct may be designed to contain a reporter gene positioned between the inverted repeats, and transfected cells positive for the reporter gene can be counted as the cells where successful transposition events occurs, which can give an estimate of the amount of the transposition events. Another non-limiting example includes sequencing of the host cell genome to examine the insertion of the cassette cargo of the transposon. In some embodiments, when the transposition efficiency of two or more different transposons is compared, the same transposase can be paired with each of the different transposons for transfection of the host cells under same or similar transfection conditions. Similar approaches can be utilized for the measurement of transposition efficiency. Other methods known to one skilled in the art may also be implemented for the comparison of transposition efficiency.

Also provided herein are methods of obtaining a hyperactive mutant SPIN transposase.

One exemplary method can comprise systemically mutating amino acids of SPIN transposase to increase a net charge of the amino acid sequence. Sometimes, the method can comprise performing systematic alanine scanning to mutate aspartic acid (D) or glutamic acid (E), which are negatively charged at a neutral pH, to alanine residues. A method can comprise performing systemic mutation to lysing (K) or arginine (R) residues, which are positively charged at a neutral pH.

Without wishing to be bound by a particular theory, increase in a net charge of the amino acid sequence at a neutral pH may increase the transposition efficiency of the SPIN transposase. Particularly, when the net charge is increased in proximity to a catalytic domain of the transposase, the transposition efficiency is expected to increase. It can be contemplated that positively charged amino acids can form points of contact with DNA target and allow the catalytic domains to act on the DNA target. It may also be contemplated that loss of these positively charged amino acids can decrease either excision or integration activity in transposases.

A mutant SPIN transposase can comprise one or more amino acid substitutions that increase a net charge at a neutral pH in comparison to SEQ ID NO: 1. Sometimes, a mutant SPIN transposase comprising one or more amino acid substitutions that increase a net charge at a neutral pH in comparison to SEQ ID NO: 1 can be hyperactive. Sometimes, the mutant SPIN transposase can comprise one or more substitutions to a positively charged amino acid, such as, but not limited to, lysine (K) or arginine (R). A mutant SPIN transposase can comprise one or more substitutions of a negatively charged amino acid, such as, but not limited to, aspartic acid (D) or glutamic acid (E), with a neutral amino acid, or a positively charged amino acid.

One non-limiting example includes a mutant SPIN transposase that comprises one or more amino acid substitutions that increase a net charge at a neutral pH within or in proximity to a catalytic domain in comparison to SEQ ID NO: 1. The catalytic domain can be the first catalytic domain or the second catalytic domain. The catalytic domain can also include both catalytic domains of the transposase.

An exemplary method of the present disclosure can comprise mutating amino acids that are predicted to be in close proximity to, or to make direct contact with, the DNA. These amino acids can be substituted amino acids identified as being conserved in other member(s) of the hAT family (e.g., other members of the Buster and/or Ac subfamilies). The amino acids predicted to be in close proximity to, or to make direct contact with, the DNA can be identified, for example, by reference to a crystal structure of SPIN transposase, predicted structures, mutational analysis, functional analysis, alignment with other members of the hAT family, or any other suitable method.

Without wishing to be bound by a particular theory, SPIN transposase, like other members of the hAT transposase family, has a DDE motif, which may be the active site that catalyzes the movement of the transposon. It is contemplated that D185, D251, and E555 make up the active site, which is a triad of acidic residues. The DDE motif may coordinate divalent metal ions and can be important in the catalytic reaction. In some embodiments, a mutant SPIN transposase can comprise one or more amino acid substitutions that increase a net charge at a neutral pH in comparison to SEQ ID NO: 1, wherein the one or more amino acids are located in proximity to D185, D251, and E555, when numbered in accordance to SEQ ID NO: 1.

In certain embodiments, a mutant SPIN transposase as provided herein does not comprise any disruption of the catalytic triad, i.e., D185, D251, and E555. A mutant SPIN transposase may not comprise any amino acid substitution at D185, D251, and E555. A mutant SPIN transposase may comprises amino acid substitution at D185, D251, and E555, but such substitution does not disrupt the catalytic activity contributed by the catalytic triad.

In some cases, the term “proximity” can refer to a measurement of a linear distance in the primary structure of the transposase. For instance, the distance between D185 and D251 in the primary structure of a wild-type SPIN transposase is 66 amino acids. In certain embodiments, the proximity can refer to a distance of about 70 to 80 amino acids. In many cases, the proximity can refer to a distance of about 80, 75, 70, 60, 50, 40, 30, 20, 10, or 5 amino acids.

In some cases, the term “proximity” can refer to a measurement of a spatial relationship in the secondary or tertiary structure of the transposase, i.e., when the transposase folds into its three-dimensional configurations. Protein secondary structure can refer to three-dimensional form of local segments of proteins. Common secondary structural elements include alpha helices, beta sheets, beta turns and omega loops. Secondary structure elements may form as an intermediate before the protein folds into its three-dimensional tertiary structure. Protein tertiary structure can refer to the three-dimensional shape of a protein. Protein tertiary structure may exhibit dynamic configurational change under physiological or other conditions. The tertiary structure will have a single polypeptide chain “backbone” with one or more protein secondary structures, the protein domains. Amino acid side chains may interact and bond in a number of ways. The interactions and bonds of side chains within a particular protein determine its tertiary structure. In many implementations, the proximity can refer to a distance of about 1 Å, about 2 Å, about 5 Å, about 8 Å, about 10 Å, about 15 Å, about 20 Å, about 25 Å, about 30 Å, about 35 Å, about 40 Å, about 50 Å, about 60 Å, about 70 Å, about 80 Å, about 90 Å, or about 100 Å.

A neutral pH can be a pH value around 7. Sometimes, a neutral pH can be a pH value between 6.9 and 7.1, between 6.8 and 7.2, between 6.7 and 7.3, between 6.6 and 7.4, between 6.5 and 7.5, between 6.4 and 7.6, between 6.3 and 7.7, between 6.2-7.8, between 6.1-7.9, between 6.0-8.0, between 5-8, or in a range derived therefrom.

Non-limiting exemplary mutant SPIN transposases that comprise one or more amino acid substitutions that increase a net charge at a neutral pH in comparison to SEQ ID NO: 1 include SPIN transposases comprising at least one of the combinations of amino acid substitutions from Table 4. A mutant SPIN transposase can comprise at least 2, at least 3, at least 4, at least 5, at least 6, at least 7, at least 8, at least 9, at least 10, at least 20, at least 30, or more of the amino acid substitutions from Table 4.

In some embodiments, a mutant SPIN transposase can comprise one or more amino acid substitutions that increase a net charge at a non-neutral pH in comparison to SEQ ID NO: 1. In some cases, the net charge is increased within or in proximity to a catalytic domain at a non-neutral pH. In many cases, the net charge is increased in proximity to D185, D251, and E555 at a non-neutral pH. The non-neutral pH can be a pH value lower than 7, lower than 6.5, lower than 6, lower than 5.5, lower than 5, lower than 4.5, lower than 4, lower than 3.5, lower than 3, lower than 2.5, lower than 2, lower than 1.5, or lower than 1. The non-neutral pH can also be a pH value higher than 7, higher than 7.5, higher than 8, higher than 8.5, higher than 9, higher than 9.5, or higher than 10.

TABLE 4

| Amino Acid of Wild-type | Amino Acid |
|---|---|
| SPIN Transposase | Substitution |
| I509 | I509R |
| I509 | I509S |
| S511 | S511N |
| L124 | L124K |
| E219 | E219K |
| A332 | A332K |
| E42 | E42R |
| A581 | A581R |
| P254 | P254K |
| P254 | P254R |
| N330 | N330H |
| E379 | E379W |

In one exemplary embodiment, a method can comprise systemically mutating amino acids in the DNA Binding and Oligomerization domain. Without wishing to be bound by a particular theory, mutation in the DNA Binding and Oligomerization domain may increase the binding affinity to DNA target and promote oligomerization activity of the transposase, which consequentially may promote transposition efficiency. More specifically, the method can comprise systemically mutating amino acids one by one within or in proximity to the DNA Binding and Oligomerization domain (e.g., amino acid 112 to 213). The method can also comprise mutating more than one amino acid within or in proximity to the DNA Binding and Oligomerization domain. The method can also comprise mutating one or more amino acids within or in proximity to the DNA Binding and Oligomerization domain, together with one or more amino acids outside the DNA Binding and Oligomerization domain.

In some embodiments, the method can comprise performing rational replacement of selective amino acid residues based on multiple sequence alignments of SPIN with other hAT family transposases (Ac, Hermes, Hobo, Tag2, Tam3, Hermes, Restless and Tol2) or with other members of Buster subfamily (e.g., AeBuster1, AeBuster2, AeBuster3, BtBuster1, BtBuster2, CfBuster1, and CfBuster2). Without being bound by a certain theory, conservancy of certain amino acids among other hAT family transposases, especially among the active ones, may indicate their importance for the catalytic activity of the transposases. Therefore, replacement of unconserved amino acids in wild-type SPIN sequence (SEQ ID NO: 1) with conserved amino acids among other hAT family may yield hyperactive mutant SPIN transposase. The method may comprise obtaining sequences of SPIN as well as other hAT family transposases: aligning the sequences and identifying the amino acids in SPIN transposase with a different conserved counterpart among the other hAT family transposases; performing site-directed mutagenesis to produce mutant SPIN transposase harboring the mutation(s).

A hyperactive mutant SPIN transposase can comprise one or more amino acid substitutions based on alignment to other members of Buster subfamily or other members of hAT family. In many cases, the one or more amino acid substitutions can be substitutions of conserved amino acid for the unconserved amino acid in wild-type SPIN sequence (SEQ ID NO: 1). Non-limiting examples of mutant SPIN transposases include SPIN transposases comprising at least one of the amino acid substitutions from Table 5. A mutant SPIN transposase can comprise at least 2, at least 3, at least 4, at least 5, at least 6, at least 7, at least 8, at least 9, at least 10, at least 20, at least 30, or more of the amino acid substitutions from Table 5.

Another exemplary method can comprise systemically mutating acidic amino acids to basic amino acids and identifying hyperactive mutant transposase.

In some cases, mutant SPIN transposase can comprise amino acid substitutions I509R, L124K, E219K, and S511N. A mutant SPIN transposase can comprise amino acid substitutions I509R and L124K. A mutant SPIN transposase can comprise amino acid substitution I509R. L124K, and E219K. A mutant SPIN transposase can comprise amino acid substitution. I509R and E219K. A mutant SPIN transposase can comprise amino acid substitution L124K, and E219K.

TABLE 5

| Amino Acid of Wild-type | Amino Acid |
|---|---|
| SPIN Transposase | Substitution |
| A332 | A332D |
| Y595 | Y595L |
| T598 | T598I |
| P254 | P254A |
| P254 | P254K |
| P254 | P254R |
| A581 | A581R |
| Q195 | Q195I |
| C41 | C41E |
| E42 | E42Q |
| A332 | A332N |
| F335 | F335L |
| V351 | V351F |
| A332 | A332K |
| A391 | A391S |
| I99 | I99V |
| I99 | I99L |
| T360 | T360M |
| T313 | T313S |
| T313 | T313A |
| L436 | L436F |
| D210 | D210E |
| N330 | N330H |
| I285 | I285A |
| M220 | M220T |
| E379 | E379W |

### Fusion Transposase

Another aspect of the present disclosure provides a fusion transposase. The fusion transposase can comprise a SPIN transposase sequence and a DNA sequence specific binding domain.

The SPIN transposase sequence of a fusion transposase can comprise an amino acid sequence of any of the mutant SPIN transposases as described herein. The SPIN transposase sequence of a fusion transposase can also comprise an amino acid sequence of a wild-type SPIN transposase having amino acid sequence SEQ ID NO: 1.

A DNA sequence specific binding domain as described herein can refer to a protein domain that is adapted to bind to a DNA molecule at a sequence region (“target sequence”) containing a specific sequence motif. For instance, an exemplary DNA sequence specific binding domain may selectively bind to a sequence motif TATA, while another exemplary DNA sequence specific binding domain may selectively bind to a different sequence motif ATGCNTAGAT (N denotes any one of A, T, G, and C).

A fusion transposase as provided herein may direct sequence specific insertion of the transposon. For instance, a DNA sequence specific binding domain may guide the fusion transposase to bind to a target sequence based on the binding specificity of the binding domain. Being bound to or restricted to a certain sequence region may spatially limit the interaction between the fusion transposase and the transposon, thereby limiting the catalyzed transposition to a sequence region in proximity to the target sequence. Depending on the size, three-dimensional configuration, and sequence binding affinity of the DNA binding domain, as well as the spatial relationship between the DNA binding domain and the SPIN transposase sequence, and the flexibility of the connection between the two domains, the distance of the actual transposition site to the target sequence may vary. Proper design of the fusion transposase configuration can direct the transposition to a desirable target genomic region.

A target genomic region for transposition can be any particular genomic region, depending on application purposes. For instance, sometimes, it is desirable to avoid transcription start sites for the transposition, which may cause undesirable, or even harmful, change in expression level of certain important endogenous gene(s) of the cell. A fusion transposase may contain a DNA sequence specific binding domain that can target the transposition to a safe harbor of the host genome. Non-limiting examples of safe harbors can include HPRT, AAVS site (e.g., AAVS1, AAVS2, ETC.), CCR5, or Rosa26. Safe harbor sites can generally refer to sites for transgene insertion whose use exert little to none disrupting effects on genome integrity of the cell or cellular health and functions.

A DNA sequence specific binding domain may be derived from, or be a variant of any DNA binding protein that has sequence-specificity. In many instances, a DNA sequence specific binding domain may comprise an amino acid sequence at least 40%, at least 50%, at least 60%, at least 70%, at least 80%, at least 90%, at least 95%, at least 98%, at least 99%, or 100% identical to a naturally occurring sequence-specific DNA binding protein. A DNA sequence specific binding domain may comprise an amino acid sequence at least 70% identical to a naturally occurring sequence-specific DNA binding protein. Non-limiting examples of a naturally occurring sequence-specific DNA binding protein include, but not limited to, transcription factors from various origins, specific-sequence nucleases, and viral replication proteins. A naturally occurring sequence-specific DNA binding protein can also be any other protein having the specific binding capability from various origins. Selection and prediction of DNA binding proteins can be conducted by various approaches, including, but not limited to, using computational prediction databases available online, like DP-Bind (http://lcg.rit.albany.edu/dp-bind/) or DNABIND (http://dnabind.szialab.org/)

The term “transcription factor” can refer to a protein that controls the rate of transcription of genetic information from DNA to messenger DNA, by binding to a specific DNA sequence. A transcription factor that can be used in a fusion transposase described herein can be based on a prokaryotic transcription factor or a eukaryotic transcription factor, as long as it confers sequence specificity when binding to the target DNA molecule. Transcription factor prediction databases such as DBD (http://www.transcriptionfactor.org) may be used for selection of appropriate transcription factor for application of the disclosure herein.

A DNA sequence specific binding domain as used herein can comprise one or more DNA binding domain from a naturally occurring transcription factor. Non-limiting examples of DNA binding domains of transcription factors include DNA binding domains that belong to families like basic helix-loop-helix, basic-leucine zipper (bZIP), C-terminal effector domain of the bipartite response regulators, AP2/ERF/GCC box, helix-turn-helix, homeodomain proteins, lambda repressor-like, srf-like (serum response factor), paired box, winged helix, zinc fingers, multi-domain Cys2His2 (C2H2) zinc fingers, Zn2/Cys6, or Zn2/Cys8 nuclear receptor zinc finger.

A DNA sequence specific binding domain can be an artificially engineered amino acid sequence that binds to specific DNA sequences. Non-limiting examples of such artificially designed amino acid sequence include sequences created based on frameworks like transcription activator like effector nucleases (TALEs) DNA binding domain, zinc finger nucleases, adeno associated virus (AAV) Rep protein, and any other suitable DNA binding proteins as described herein.

Natural TALEs are proteins secreted by Xanthomonas bacteria to aid the infection of plant species. Natural TALEs can assist infections by binding to specific DNA sequences and activating the expression of host genes. In general, TALE proteins consist of a central repeat domain, which determines the DNA targeting specificity and can be rapidly synthesized de novo. TALEs have a modular DNA-binding domain (DBD) containing repetitive sequences of residues. In some TALEs, each repeat region contains 34 amino acids. The term “TALE domain” as used herein can refer to the modular DBD of TALEs. A pair of residues at the 12th and 13th position of each repeat region can determine the nucleotide specificity and are referred to as the repeat variable diresidue (RVD). The last repeat region, termed the half-repeat, is typically truncated to 20 amino acids. Combining these repeat regions allows synthesizing sequence-specific synthetic TALEs. The C-terminus typically contains a nuclear localization signal (NLS), which directs a TALE to the nucleus, as well as a functional domain that modulates transcription, such as an acidic activation domain (AD). The endogenous NLS can be replaced by an organism-specific localization signal. For example, an NLS derived from the simian virus 40 large T-antigen can be used in mammalian cells. The RVDs HD, NG, NI, and NN target C, T. A, and G/A, respectively. A list of RVDs and their binding preferences under certain circumstances for nucleotides can be found in Table 6. Additional TALE RVDs can also be used for custom degenerate TALE-DNA interactions. For example, NA has high affinity for all four bases of DNA. Additionally, N*, where * is an RVD with a deletion in the 13th residue, can accommodate all letters of DNA including methylated cytosine. Also S* may have the ability to bind to any DNA nucleotide.

A number of online tools are available for designing TALEs to target a specific DNA sequence, for example TALE-NT (https://tale-nt.cac.cornell.edu/), Mojo hand (http://www.talendesign.org/). Commercially available kits may also assist in creating custom assembly of TALE repeat regions between the N and C-terminus of the protein. These methods can be used to assemble custom DBDs, which are then cloned into an expression vector containing a functional domain, e.g., SPIN transposase sequence.

TABLE 6
RVD Binding Preference

nucleotides

| RVD | A | G | C | T |
|---|---|---|---|---|
| NN | medium | medium | | |
| NK | | weak | | |
| NI | medium | | | |
| NG | | | | weak |
| HD | | | medium | |
| NS | weak | medium | weak | weak |
| NG | | | | weak |
| N* | | | weak | weak |
| HN | weak | medium | | |
| NT | weak | medium | | |
| NP | weak | | weak | medium |
| NH | | medium | | |
| SN | | weak | | |
| SH | | weak | | |
| NA | weak | strong | weak | weak |
| IG | | | | weak |
| H* | poor | poor | weak | poor |
| ND | | | weak | |
| HI | medium | | | |
| HG | | | | weak |
| NC | | | | weak |
| NQ | | weak | | |
| SS | | weak | | |
| SN | | weak | | |
| S* | medium | medium | strong | medium |
| NV | weak | medium | poor | poor |
| HH | poor | poor | poor | poor |
| YG | poor | poor | poor | poor |

TALEs can be synthesized de novo in the laboratory, for example, by combining digestion and ligation steps in a Golden Gate reaction with type II restriction enzymes. Alternatively, TALE can be assembled by a number of different approaches, including, but not limited to, Ligation-Independent Cloning (LIC), Fast Ligation-based Automatable Solid-phase High-throughput (FLASH) assembly, and Iterative-Capped Assembly (ICA).

Zinc fingers (ZF) are ˜30 amino acids that can bind to a limited combination of ˜3 nucleotides. The C2H2 ZF domain may be the most common type of ZF and appears to be one of the most abundantly expressed proteins in eukaryotic cells. ZFs are small, functional and independently folded domains coordinated with zinc molecules in their structure. Amino acids in each ZF can have affinity towards specific nucleotides, causing each finger to selectively recognize 3-4 nucleotides of DNA. Multiple ZFs can be arranged into a tandem array and recognize a set of nucleotides on the DNA. By using a combination of different zinc fingers, a unique DNA sequence within the genome can be targeted. Different ZFPs of various lengths can be generated, which may allow for recognition of almost any desired DNA sequence out of the possible 64 triplet subsites.

Zinc fingers to be used in connection with the present disclosure can be created using established modular assembly fingers, such as a set of modular assembly finger domains developed by Barbas and colleagues, and also another set of modular assembly finger domains by ToolGen. Both set of domains cover all 3 bp GNN, most ANN, many CNN and some TNN triplets (where N can be any of the four nucleotides). Both have a different set of fingers, which allows for searching and coding different ZF modules as needed. A combinatorial selection-based oligomerized pool engineering (OPEN) strategy can also be employed to minimize context-dependent effects of modular assembly involving the position of a finger in the protein and the sequence of neighboring fingers. OPEN ZF arrays are publicly available from the Zinc Finger Consortium Database.

AAV Rep DNA-binding domain is another DNA sequence specific binding domain that can be used in connection with the subject matter of the present disclosure. Viral cis-acting inverted terminal repeats (ITRs), and the trans-acting viral Rep proteins (Rep) are believed to be the factors mediating preferential integration of AAV into AAVS1 site of the host genome in the absence of a helper virus. AAV Rep protein can bind to specific DNA sequence in the AAVS1 site. Therefore, a site-specific DNA-binding domain can be fused together with a SPIN transposase domain as described herein.

A fusion transposase as provided herein can comprise a SPIN transposase sequence and a tag sequence. A tag sequence as provide herein can refer to any protein sequence that can be used as a detection tag of the fusion protein, such as, but not limited to, reporter proteins and affinity tags that can be recognized by antibodies. Reporter proteins include, but not limited to, fluorescent proteins (e.g., GFP, RFP, mCherry, YFP), β-galactosidase (β-gal), alkaline phosphatase (AP), chloramphenicol acetyl transferase (CAT), horseradish peroxidase (HRP). Non-limiting examples of affinity tags include polyhistidine (His tag). Glutathione S-Transferase (GST), Maltose Binding Protein (MBP), Calmodulin Binding Peptide (CBP), intein-chitin binding domain (intein-CBD), Streptavidin/Biotin-based tags, Epitope tags like FLAG, HA, c-myc, T7, Glu-Glu and many others.

A fusion transposase as provided herein can comprise a SPIN transposase sequence and a DNA sequence specific binding domain or a tag sequence fused together without any intermediate sequence (e.g., “back-to-back”). In some cases, a fusion transposase as provided herein can comprise a SPIN transposase sequence and a DNA sequence specific binding domain or a tag sequence joined by a linker sequence. In an exemplary fusion transposase, a linker may serve primarily as a spacer between the first and second polypeptides. A linker can be a short amino acid sequence to separate multiple domains in a single polypeptide. A linker sequence can comprise linkers occurring in natural multi-domain proteins. In some instances, a linker sequence can comprise linkers artificially created. The choice of linker sequence may be based on the application of the fusion transposase. A linker sequence can comprise 3, 4, 5, 6, 7, 8, 9, 10, or more amino acids. In some embodiments, the linker sequence may comprise at least 3, at least 4, at least 5, at least 6, at least 7, at least 8, at least 9, at least 10, at least 15, at least 20, or at least 50 amino acids. In some embodiments, the linker sequence can comprise at most 4, at most 5, at most 6, at most 7, at most 8, at most 9, at most 10, at most 11, at most 12, at most 15, at most 20, at most 30, at most 40, at most 50, or at most 100 amino acids. In certain cases, it may be desirable to use flexible linker sequences, such as, but not limited to, stretches of Gly and Ser residues (“GS” linker) like (GGGGS)n (n=2-8), (Gly)₈, GSAGSAAGSGEF, (GGGGS)₄. Sometimes, it may be desirable to use rigid linker sequences, such as, but not limited to, (EAAAK)n (n=2-7), Pro-rich sequences like (XP)n, with X designating any amino acid.

In an exemplary fusion transposase provided herein, a SPIN transposase sequence can be fused to the N-terminus of a DNA sequence specific binding domain or a tag sequence. Alternatively, a SPIN transposase sequence can be fused to the C-terminus of a DNA sequence specific binding domain or a tag sequence. In some embodiments, a third domain sequence or more of other sequences can be present in between the SPIN transposase and the DNA sequence specific binding domain or the tag sequence, depending on the application of the fusion transposase.

### Dual Transposase System

In some aspects, the disclosure further provides a dual transposase system that comprises two different transposases, one of which desirably is the mutant SPIN transposase described herein. The second transposase may be any suitable transposase used for mutagenesis of eukaryotic genomes, so long as the second transposase recognizes a different transposon than is recognized by the mutant SPIN transposase. In some embodiments, the second transposase is a Class II transposase, such as a a hAT transposase. The hAT transposase may be a transposase of the Ac, Sleeping Beauty, PiggyBac, or Buster subfamilies. For example, the second transposase may be a TcBuster transposase, such as a mutant TcBuster transposase. The mutant TcBuster transposase may have an increased transposition efficiency as compared to a wild-type TcBuster transposase. Exemplary mutant TcBuster transposases that may be used in combination with the SPIN transposase disclosed herein are described in, e.g., U.S. Pat. No. 10,227,574. In some embodiments, a mutant TcBuster transposase may comprise one or more amino acid substitutions in comparison to a wild-type TcBuster transposase (SEQ ID NO: 12). For example, a mutant TcBuster transposase may comprise an amino acid sequence that is at least 70% identical to full-length SEQ ID NO: 12 and an amino acid substitution of V377T, E469K, D189A, K573E, E578L, or any combination thereof, when numbered in accordance with SEQ ID NO: 12. In other embodiments, a mutant TcBuster transposase may comprise an amino acid sequence that is at least 70% identical to full-length SEQ ID NO: 12 and an amino acid substitution of D58E, N85S, D99A, E247K, V377T, E469K, or any combination thereof, when numbered in accordance with SEQ ID NO: 12. In other embodiments, a mutant TcBuster transposase may comprise an amino acid sequence that is at least 70% identical to full-length SEQ ID NO: 12 and an amino acid substitution of N85S, D99A, E247K, V377T, R382K, E469K, or any combination thereof, when numbered in accordance with SEQ ID NO: 12. In yet other embodiments, a mutant TcBuster may comprise an amino acid sequence that is at least 70% identical to full-length SEQ ID NO: 12 and an amino acid substitution of N85S, D99A, E247K, V377T, E469K, or any combination thereof, when numbered in accordance with SEQ ID NO: 12.

The disclosure also provides a method of genome editing, which comprises introducing into a cell: (a) the mutant SPIN transposase of any one of claims 1-19, (b) a second transposase (c) a first transposon recognizable by the mutant SPIN transposase but not the second transposase, and (d) a second transposon recognizable by the second transposase but not the mutant SPIN transposase. Transposons recognizable by the mutant SPIN transposase are described herein. When the second transposase of the dual transposase system is a hAT transposase, any transposon recognizable by the particular hAT transposase may be employed. For example, when the second transposase is a mutant TcBuster transposase, such as a mutant TcBuster transposase described above or otherwise known in the art, exemplary transposons that may be employed are described in, e.g., U.S. Pat. No. 10,227,574. The mutant SPIN transposase and the second transposase may be introduced into a cell simultaneously or sequentially in any order.

The dual transposase system and method provided herein may be used in a variety of applications. In some embodiments, for example, a dual transposase system may be used to develop stable cell lines expressing multiple non-native genes. In this respect, using single transposase systems to generate stable cell lines that overexpress multiple genes can be limited by remobilization of the transposon and excision of previously integrated genes of interest. In contrast, a dual transposase system allows for more flexible gene introduction into cells for the production of, e.g., recombinant proteins, monoclonal antibodies, and viruses or virus subunits (e.g., lentiviruses, adeno-associated virus (AAV), and adenovirus).

### SPIN Transposon

Another aspect of the present disclosure provides a SPIN transposon that comprises a cassette cargo positioned between two inverted repeats. A SPIN transposon can be recognized by a SPIN transposase as described herein, e.g., a SPIN transposase can recognize the SPIN transposon and catalyze transposition of the SPIN transposon into a DNA sequence.

The terms “inverted repeats”, “terminal inverted repeats”, “inverted terminal repeats”, as used interchangeably herein, can refer to short sequence repeats flanking the transposase gene in a natural transposon or a cassette cargo in an artificially engineered transposon. The two inverted repeats are generally required for the mobilization of the transposon in the presence of a corresponding transposase. Inverted repeats as described herein may contain one or more direct repeat (DR) sequences. These sequences usually are embedded in the terminal inverted repeats (TIRs) of the elements. The term “cargo cassette” as used herein can refer to a nucleotide sequence other than a native nucleotide sequence between the inverted repeats that contains the SPIN transposase gene. A cargo cassette can be artificially engineered.

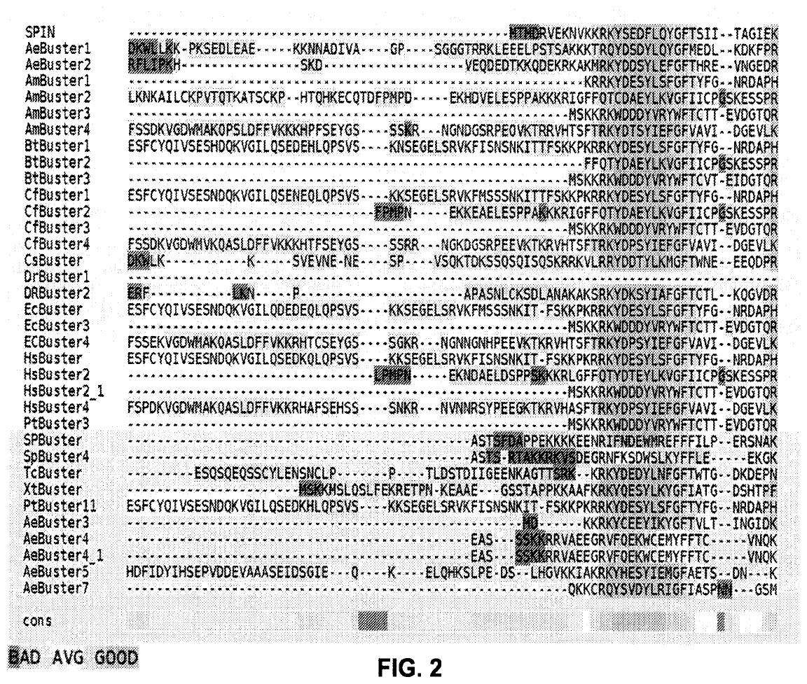

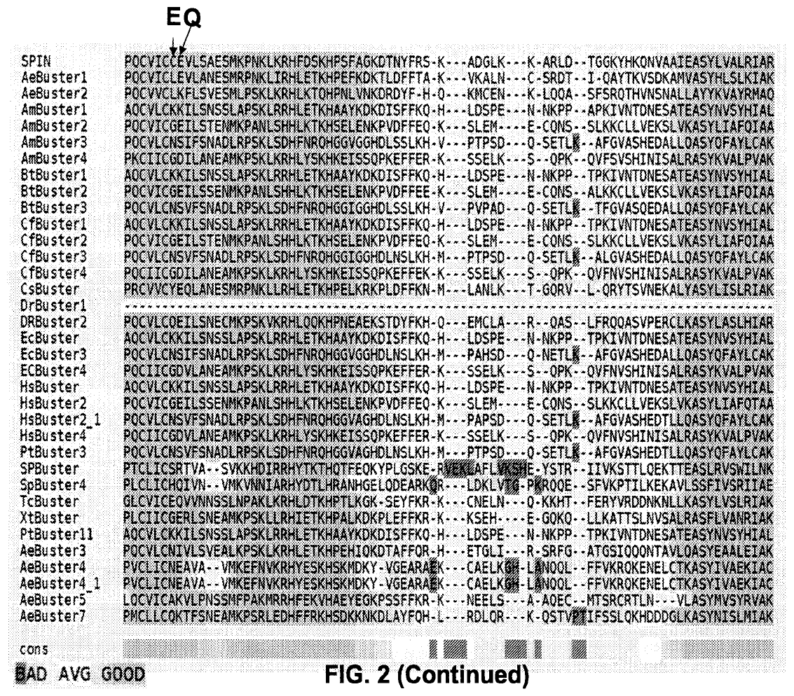

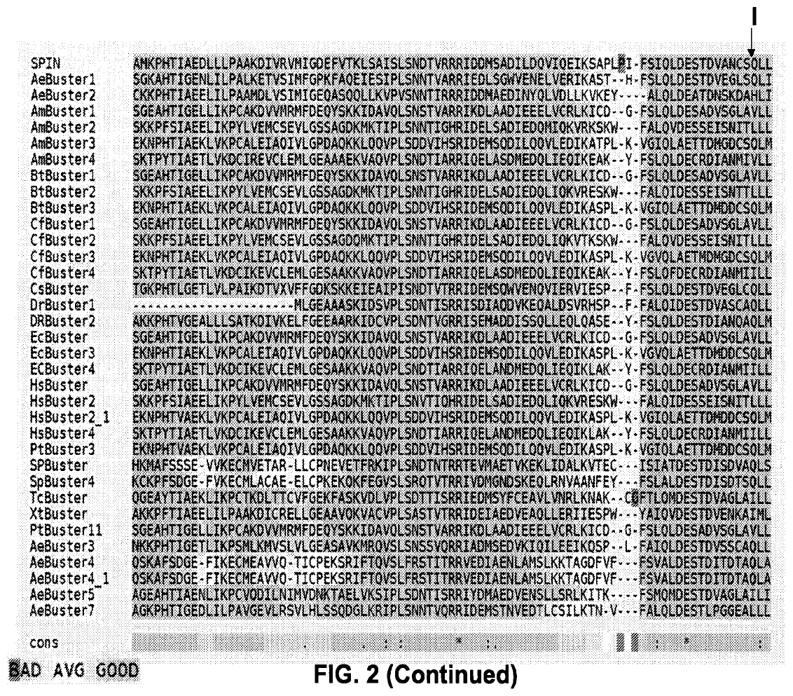

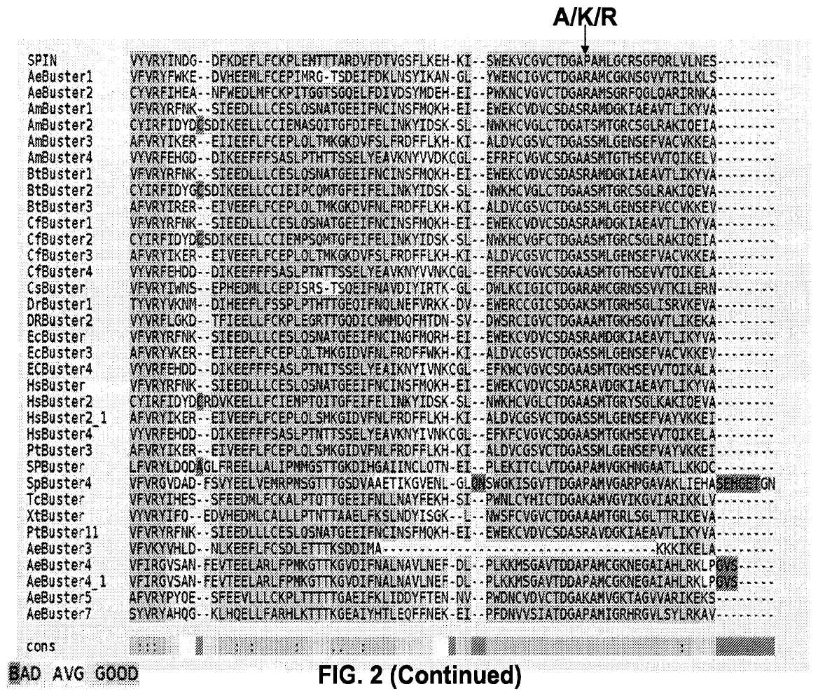

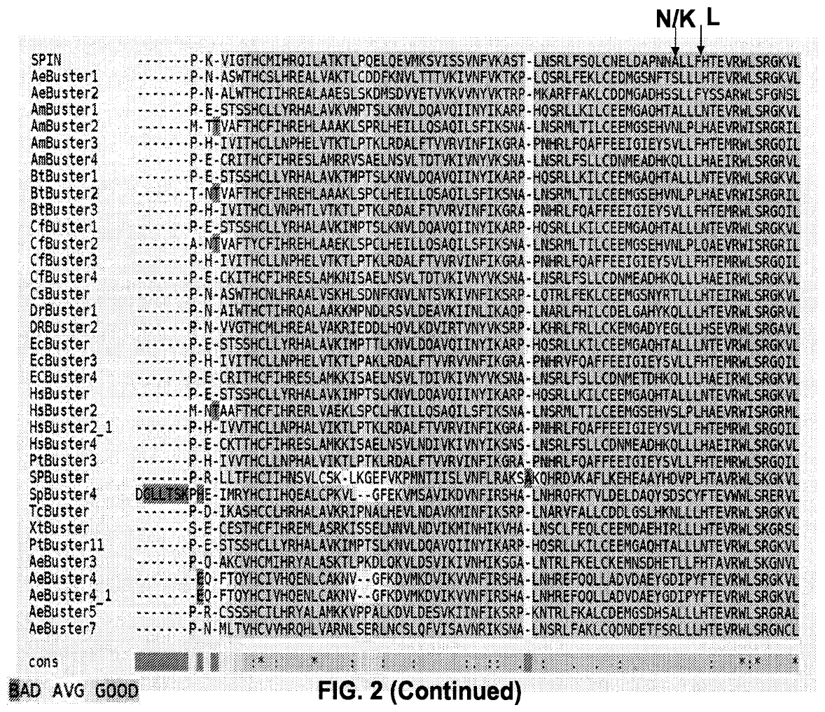

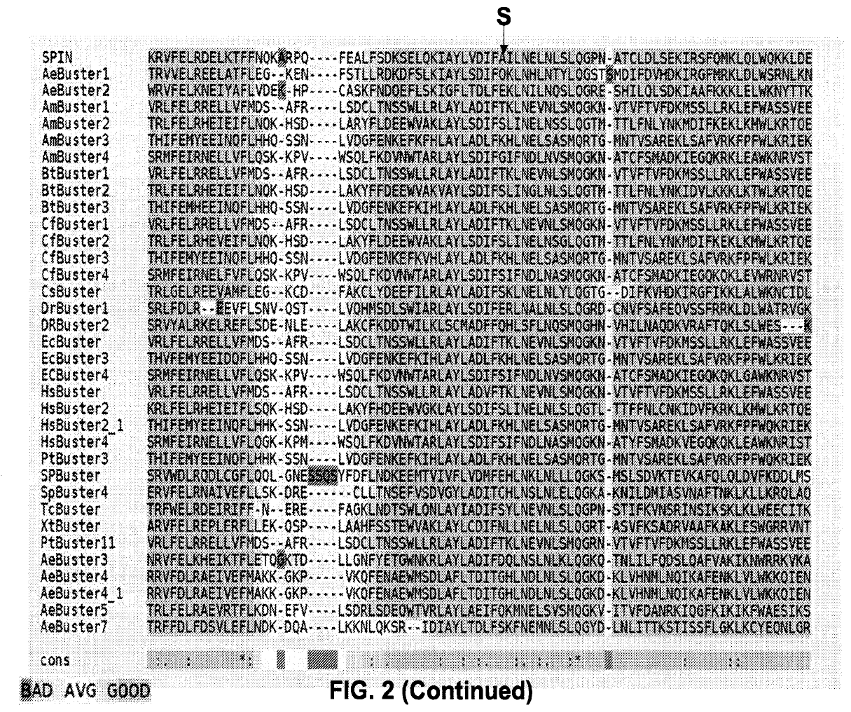

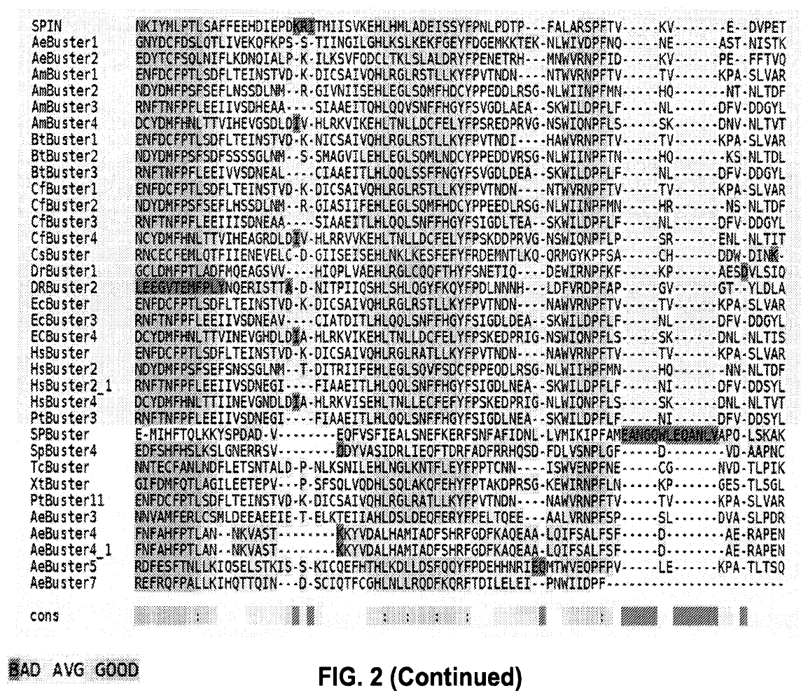

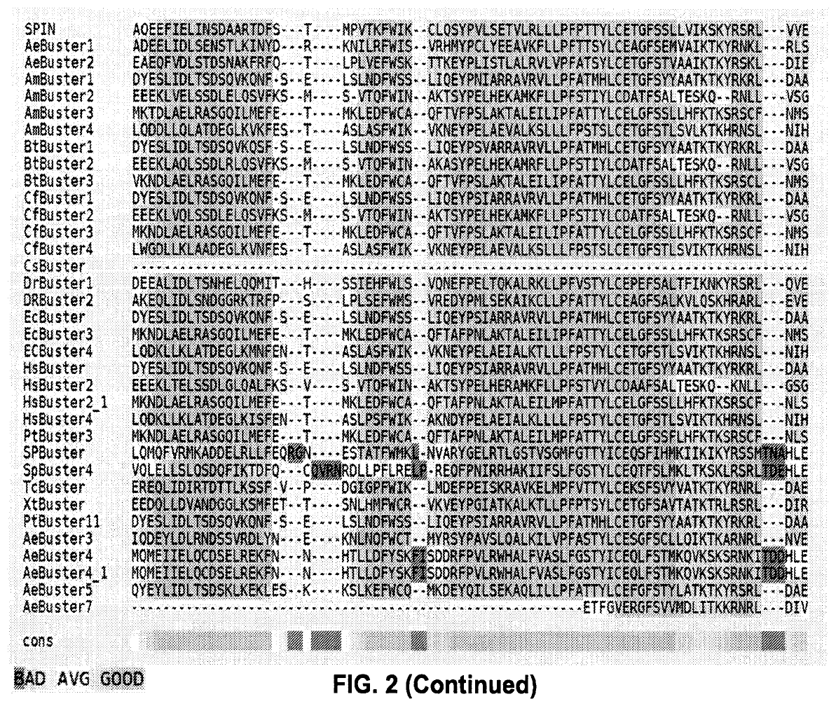

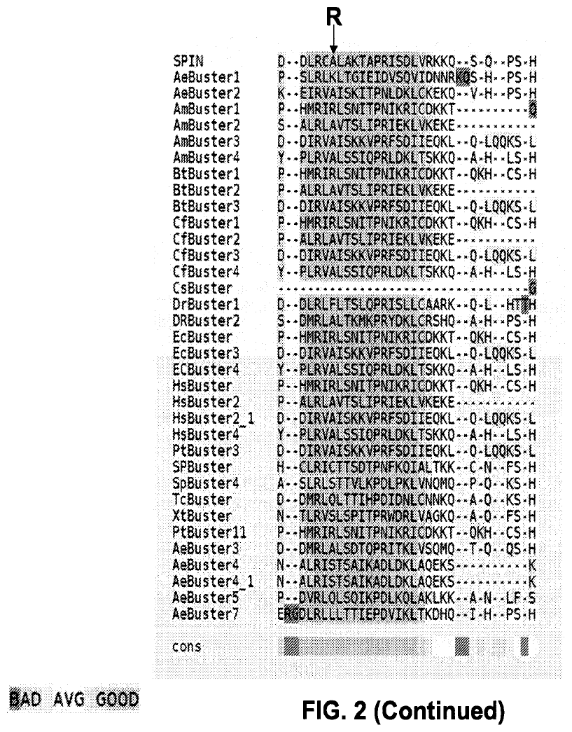

A transposon described herein may contain a cargo cassette flanked by IR/DR sequences. In some embodiments, at least one of the repeats contains at least one direct repeat. As shown in FIGS. 1 and 2, a transposon may contain a cargo cassette flanked by ITR-L-Seq (SEQ ID NO: 3) and ITR-R-Seq (SEQ ID NO: 4). In many cases, a left inverted repeat can comprise a sequence at least 40%, at least 50%, at least 60%, at least 70%, at least 80%, at least 90%, at least 95%, at least 98%, at least 99%, or 100% identical to ITR-L-Seq (SEQ ID NO: 3). Sometimes, a right inverted repeat can comprise a sequence at least 40%, at least 50%, at least 60%, at least 70%, at least 80%, at least 90%, at least 95%, at least 98%, at least 99%, or 100% identical to ITR-R-Seq (SEQ ID NO: 4). In other cases, a right inverted repeat can comprise a sequence at least 40%, at least 50%, at least 60%, at least 70%, at least 80%, at least 90%, at least 95%, at least 98%, at least 99%, or 100% identical to ITR-L-Seq (SEQ ID NO: 3). Sometimes, a left inverted repeat can comprise a sequence at least 40%, at least 50%, at least 60%, at least 70%, at least 80%, at least 90%, at least 95%, at least 98%, at least 99%, or 100% identical to ITR-R-Seq (SEQ ID NO: 4). The terms “left” and “right”, as used herein, can refer to the 5′ and 3′ sides of the cargo cassette on the sense strand of the double strand transposon, respectively. A transposon may contain a cargo cassette flanked by two inverted repeats that have different nucleotide sequences, or a combination of the various sequences known to one skilled in the art. At least one of the two inverted repeats of a transposon described herein may contain a sequence that is at least 40%, at least 50%, at least 60%, at least 70%, at least 80%, at least 90%, at least 95%, at least 98%, at least 99%, or 100% identical to SEQ ID NO: 3 or SEQ ID NO: 4. At least one of inverted repeats of a transposon described herein may contain a sequence that is at least 80% identical to SEQ ID NO: 3 or 4. The choice of inverted repeat sequences may vary depending on the expected transposition efficiency, the type of cell to be modified, the transposase to use, and many other factors.

In many implementations, minimally sized transposon vector inverted terminal repeats that conserve genomic space may be used. The ITRs of hAT family transposons diverge greatly with differences in right-hand and left-hand ITRs. In many cases, smaller ITRs consisting of just 100-200 nucleotides are as active as the longer native ITRs in hAT transposon vectors. These sequences may be consistently reduced while mediating hAT family transposition. These shorter ITRs can conserve genomic space within hAT transposon vectors.

The inverted repeats of a transposon provided herein can be about 50 to 2000 nucleotides, about 50 to 1000 nucleotides, about 50 to 800 nucleotides, about 50 to 600 nucleotides, about 50 to 500 nucleotides, about 50 to 400 nucleotides, about 50 to 350 nucleotides, about 50 to 300 nucleotides, about 50 to 250 nucleotides, about 50 to 200 nucleotides, about 50 to 180 nucleotides, about 50 to 160 nucleotides, about 50 to 140 nucleotides, about 50 to 120 nucleotides, about 50 to 110 nucleotides, about 50 to 100 nucleotides, about 50 to 90 nucleotides, about 50 to 80 nucleotides, about 50 to 70 nucleotides, about 50 to 60 nucleotides, about 75 to 750 nucleotides, about 75 to 450 nucleotides, about 75 to 325 nucleotides, about 75 to 250 nucleotides, about 75 to 150 nucleotides, about 75 to 95 nucleotides, about 100 to 500 nucleotides, about 100 to 400 nucleotides, about 100 to 350 nucleotides, about 100 to 300 nucleotides, about 100 to 250 nucleotides, about 100 to 220 nucleotides, about 100 to 200 nucleotides, or in any range derived therefrom.

In some cases, a cargo cassette can comprise a promoter, a transgene, or a combination thereof. In cargo cassettes comprising both a promoter and a transgene, the expression of the transgene can be directed by the promoter. A promoter can be any type of promoter available to one skilled in the art. Non-limiting examples of the promoters that can be used in a SPIN transposon include EFS, CMV, MND, EF1α, CAGGs, PGK, UBC, U6, H1, and Cumate. The choice of a promoter to be used in a SPIN transposition would depend on a number of factors, such as, but not limited to, the expression efficiency of the promoter, the type of cell to be genetically modified, and the desired transgene expression level.

A transgene in a SPIN transposon can be any gene of interest and available to one skilled in the art. A transgene can be derived from, or a variant of, a gene in nature, or can be artificially designed. A transgene can be of the same species origin as the cell to be modified, or from different species. A transgene can be a prokaryotic gene, or a eukaryotic gene. Sometimes, a transgene can be a gene derived from a non-human animal, a plant, or a human being. A transgene can comprise introns. Alternatively, a transgene may have introns removed or not present.

In some embodiments, a transgene can code for a protein. Exemplary proteins include, but are not limited to, a cellular receptor, an immunological checkpoint protein, a cytokine, or any combination thereof. Sometimes, a cellular receptor as described herein can include, but not limited to a T cell receptor (TcR), a B cell receptor (BcR), a chimeric antigen receptor (CAR), or any combination thereof.

A cargo cassette as described herein may not contain a transgene coding for any type of protein product, but that is useful for other purposes. For instance, a cargo cassette may be used for creating frameshift in the insertion site, for example, when it is inserted in an exon of a gene in the host genome. This may lead to a truncation of the gene product or a null mutation. Sometimes, a cargo cassette may be used for replacing an endogenous genomic sequence with an exogenous nucleotide sequence, thereby modifying the host genome.

A transposon described herein may have a cargo cassette in either forward or reverse direction. In many cases, a cargo cassette has its own directionality. For instance, a cargo cassette containing a transgene would have a 5′ to 3′ coding sequence. A cargo cassette containing a promoter and a gene insertion would have promoter on the 5′ site of the gene insertion. The term “forward direction”, as used herein, can refer to the situation where a cargo cassette maintains its directionality on the sense strand of the double strand transposon. The term “reverse direction”, as used herein, can refer to the situation where a cargo cassette maintains its directionality on the antisense strand of the double strand transposon.

### Systems for Genome Editing and Methods of Use

Another aspect of the present disclosure provides a system for genome editing. A system can comprise a SPIN transposase and a SPIN transposon. A system can be used to edit a genome of a host cell, disrupting or modifying an endogenous genomic region of the host cell, inserting an exogenous gene into the host genome, replacing an endogenous nucleotide sequence with an exogenous nucleotide sequence or any combination thereof.

A system for genome editing can comprise a mutant SPIN transposase or fusion transposase as described herein, and a transposon recognizable by the mutant SPIN transposase or the fusion transposase. A mutant SPIN transposase or the fusion transposase can be provided as a purified protein. Protein production and purification technologies are known to one skilled in the art. The purified protein can be kept in a different container than the transposon, or they can be kept in the same container.

In many cases, a system for genome editing can comprise a polynucleotide encoding a mutant SPIN transposase or fusion transposase as described herein, and a transposon recognizable by the mutant SPIN transposase or the fusion transposase. Sometimes, a polynucleotide of the system can comprise DNA that encodes the mutant SPIN transposase or the fusion transposase. Alternatively or additionally, a polynucleotide of the system can comprise messenger RNA (mRNA) that encodes the mutant SPIN transposase or the fusion transposase. The mRNA can be produced by a number of approaches well known to one of ordinary skills in the art, such as, but not limited to, in vivo transcription and RNA purification, in vitro transcription, and de novo synthesis. In many cases, the mRNA can be chemically modified. The chemically modified mRNA may be resistant to degradation than unmodified or natural mRNAs or may degrade more quickly. In many cases, the chemical modification of the mRNA may render the mRNA being translated with more efficiency. Chemical modification of mRNAs can be performed with well-known technologies available to one skilled in the art, or by commercial vendors.

For many applications, safety dictates that the duration of hAT transposase expression be only long enough to mediate safe transposon delivery. Moreover, a pulse of hAT transposase expression that coincides with the height of transposon vector levels can achieve maximal gene delivery. The implementations are made using available technologies for the in vitro transcription of RNA molecules from DNA plasmid templates. The RNA molecules can be synthesized using a variety of methods for in vitro (e.g., cell free) transcription from a DNA copy. Methods to do this have been described and are commercially available. For example, the mMessage Machine in vitro transcription kit available through life technologies.

There are also a number of companies that can perform in vitro transcription on a fee for service basis. We have also found that that chemically modified RNAs for hAT expression work especially well for gene transfer. These chemically modified RNAs do not induce cellular immune responses and RNA generated using proprietary methods that also avoid the cellular immune response. These RNA preparations remove RNA dimers (Clean-Cap) and cellular reactivity (pseudouridine incorporation) produce better transient gene expression in human T cells without toxicity in our hands (data not shown). The RNA molecules can be introduced into cells using any of many described methods for RNA transfection, which is usually non-toxic to most cells. Methods to do this have been described and are commercially available. For example, the Amaxa nucleofector, Neon electroporator, and the Maxcyte platforms.

A transposon as described herein may be present in an expression vector. In many cases, the expression vector can be DNA plasmid. Sometimes, the expression vector can be a mini-circle vector. The term “mini-circle vector” as used herein can refer to small circular plasmid derivative that is free of most, if not all, prokaryotic vector parts (e.g., control sequences or non-functional sequences of prokaryotic origin). Under circumstances, the toxicity to the cells created by transfection or electroporation can be mitigated by using the “mini-circles” as described herein.

A mini-circle vector can be prepared by well-known molecular cloning technologies available. First, a ‘parental plasmid’ (bacterial plasmid with insertion, such as transposon construct) in bacterial, such as E. coli, can be produced, which can be followed by induction of a site-specific recombinase. These steps can then be followed by the excision of prokaryotic vector parts via two recombinase-target sequences at both ends of the insert, as well as recovery of the resulting mini-circle vector. The purified mini-circle can be transferred into the recipient cell by transfection or lipofection and into a differentiated tissue by, for instance, jet injection. A mini-circle containing SPIN transposon can have a size about 1.5 kb, about 2 kb, about 2.2 kb, about 2.4 kb, about 2.6 kb, about 2.8 kb, about 3 kb, about 3.2 kb, about 3.4 kb, about 3.6 kb, about 3.8 kb, about 4 kb, about 4.2 kb, about 4.4 kb, about 4.6 kb, about 4.8 kb, about 5 kb, about 5.2 kb, about 5.4 kb, about 5.6 kb, about 5.8 kb, about 6 kb, about 6.5 kb, about 7 kb, about 8 kb, about 9 kb, about 10 kb, about 12 kb, about 25 kb, about 50 kb, or a value between any two of these numbers. Sometimes, a mini-circle containing SPIN transposon as provided herein can have a size at most 2.1 kb, at most 3.1 kb, at most 4.1 kb, at most 4.5 kb, at most 5.1 kb, at most 5.5 kb, at most 6.5 kb, at most 7.5 kb, at most 8.5 kb, at most 9.5 kb, at most 11 kb, at most 13 kb, at most 15 kb, at most 30 kb, or at most 60 kb.

In certain embodiments, a system as described herein may contain a polynucleotide encoding a mutant SPIN transposase or fusion transposase as described herein, and a transposon, which are present in a same expression vector, e.g., plasmid.

Another aspect of the present disclosure provides a method of genetic engineering. A method of genetic engineering can comprise introducing into a cell a SPIN transposase and a transposon recognizable by the SPIN transposase. A method of genetic engineering can also be performed in a cell-free environment. A method of genetic engineering in a cell-free environment can comprise combining a SPIN transposase, a transposon recognizable by the transposase, and a target nucleic acid into a container, such as a well or tube.

A method described herein can comprises introducing into a cell a mutant SPIN transposase provided herein and a transposon recognizable by the mutant SPIN transposase. A method of genome editing can comprise: introducing into a cell a fusion transposase provided herein and a transposon recognizable by the fusion transposase.

The mutant SPIN transposase or the fusion transposase can be introduced into the cell either as a protein or via a polynucleotide that encodes for the mutant SPIN transposase or the fusion transposase. The polynucleotide, as discussed above, can comprise a DNA or an mRNA that encodes the mutant SPIN transposase or the fusion transposase.

In many instances, the SPIN transposase or the fusion transposase can be transfected into a host cell as a protein, and the concentration of the protein can be at least 0.05 nM, at least 0.1 nM, at least 0.2 nM, at least 0.5 nM, at least 1 nM, at least 2 nM, at least 5 nM, at least 10 nM, at least 50 nM, at least 100 nM, at least 200 nM, at least 500 nM, at least 1 μM, at least 2 μM, at least 5 μM, at least 7.5 μM, at least 10 μM, at least 15 μM, at least 20 μM, at least 25 μM, at least 50 μM, at least 100 μM, at least 200 μM, at least 500 μM, or at least 1 μM. Sometimes, the concentration of the protein can be around 1 μM to around 50 μM, around 2 μM to around 25 μM, around 5 μM to around 12.5 μM, or around 7.5 μM to around 10 μM.

In many cases, the SPIN transposase or the fusion transposase can be transfected into a host cell through a polynucleotide, and the concentration of the polynucleotide can be at least about 5 ng/ml, 10 ng/ml, 20 ng/ml, 40 ng/ml, 50 ng/ml, 60 ng/ml, 80 ng/ml, 100 ng/ml, 120 ng/ml, 150 ng/ml, 180 ng/ml, 200 ng/ml, 220 ng/ml, 250 ng/ml, 280 ng/ml, 300 ng/ml, 500 ng/ml, 750 ng/ml, 1 μg/ml, 2 μg/ml, 3 μg/ml, 5 μg/ml, 50 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml 650 μg/ml, 700 μg/ml, 750 μg/ml, or 800 μg/ml. Sometimes, the concentration of the polynucleotide can be between about 5-25 μg/ml, 25-50 μg/ml, 50-100 μg/ml, 100-150 μg/ml, 150-200 μg/ml, 200-250 μg/ml, 250-500 μg/ml, 5-800 μg/ml, 200-800 μg/ml, 250-800 μg/mil, 400-800 μg/ml, 500-800 μg/ml, or any range derivable therein. In many cases, the transposon is present in a separate expression vector than the transposase, and the concentration of the transposon can be at least about 5 ng/ml, 10 ng/ml, 20 ng/ml, 40 ng/ml, 50 ng/ml, 60 ng/ml, 80 ng/ml, 100 ng/ml, 120 ng/ml, 150 ng/ml, 180 ng/ml, 200 ng/ml, 220 ng/ml, 250 ng/ml, 280 ng/ml, 300 ng/ml, 500 ng/ml, 750 ng/ml, 1 μg/ml, 2 μg/ml, 3 μg/ml, 5 μg/ml, 50 μg/ml, 100 μg/ml, 150 μg/ml, 200 μg/ml, 250 μg/ml, 300 μg/ml, 350 μg/mi, 400 μg/ml, 450 μg/ml, 500 μg/ml, 550 μg/ml, 600 μg/ml, 650 μg/ml, 700 μg/ml, 750 μg/ml, or 800 μg/ml. Sometimes, the concentration of the transposon can be between about 5-25 μg/ml, 25-50 μg/ml, 50-100 μg/ml, 100-150 μg/ml, 150-200 μg/ml, 200-250 μg/ml, 250-500 μg/ml, 5-800 μg/ml, 200-800 μg/ml, 250-800 μg/ml, 400-800 μg/ml, 500-800 μg/ml, or any range derivable therein. It is possible the ratio of the transposon versus the polynucleotide coding for the transposase is at most 10000, at most 5000, at most 1000, at most 500, at most 200, at most 100, at most 50, at most 20, at most 10, at most 5, at most 2, at most 1, at most 0.1, at most 0.05, at most 0.01, at most 0.001, at most 0.0001, or any number in between any two thereof.

In some other cases, the transposon and the polynucleotide coding for the transposase are present in the same expression vector, and the concentration of the expression vector containing both transposon and the polynucleotide encoding transposase can be at least about 5 ng/ml, 10 ng/ml, 20 ng/ml, 40 ng/ml, 50 ng/ml, 60 ng/ml, 80 ng/ml, 100 ng/ml, 120 ng/ml, 150 ng/ml, 180 ng/ml, 200 ng/ml, 220 ng/ml, 250 ng/ml, 280 ng/ml, 300 ng/ml, 500 ng/ml, 750 ng/ml, 1 μg/ml, 2 μg/ml, 3 μg/ml, 5 μg/ml, 50 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, or 800 μg/ml. Sometimes, the concentration of the expression vector containing both transposon and the polynucleotide encoding transposase can be between about 5-25 μg/ml, 25-50 μg/ml, 50-100 μg/ml, 100-150 μg/ml, 150-200 μg/ml, 200-250 μg/ml, 250-500 μg/ml, 5-800 μg/ml, 200-800 μg/ml, 250-800 μg/ml, 400-800 μg/ml, 500-800 μg/ml, or any range derivable therein.

In some cases, the amount of polynucleic acids that may be introduced into the cell by electroporation may be varied to optimize transfection efficiency and/or cell viability. In some cases, less than about 100 μg of nucleic acid may be added to each cell sample (e.g., one or more cells being electroporated). In some cases, at least about 100 μg, at least about 200 μg, at least about 300 μg, at least about 400 μg, at least about 500 μg, at least about 600 μg, at least about 700 μg, at least about 800 μg, at least about 900 μg, at least about 1 microgram, at least about 1.5 μg, at least about 2 μg, at least about 2.5 μg, at least about 3 μg, at least about 3.5 μg, at least about 4 μg, at least about 4.5 μg, at least about 5 μg, at least about 5.5 μg, at least about 6 μg, at least about 6.5 sg, at least about 7 μg, at least about 7.5 μg, at least about 8 μg, at least about 8.5 μg, at least about 9 μg, at least about 9.5 μg, at least about 10 μg, at least about 11 μg, at least about 12 μg, at least about 13 μg, at least about 14 μg, at least about 15 μg, at least about 20 μg, at least about 25 μg, at least about 30 μg, at least about 35 μg, at least about 40 μg, at least about 45 μg, or at least about 50 μg, of nucleic acid may be added to each cell sample (e.g., one or more cells being electroporated). For example, 1 microgram of dsDNA may be added to each cell sample for electroporation. In some cases, the amount of polynucleic acids (e.g., dsDNA) required for optimal transfection efficiency and/or cell viability may be specific to the cell type.

The subject matter disclosed herein may find use in genome editing of a wide range of various types of host cells. In preferred embodiments, the host cells may be from eukaryotic organisms. In some embodiments, the cells may be from a mammal origin. In some embodiments, the cells may be from a human origin.

In general, the cells may be from an immortalized cell line or primary cells.

The terms “cell line” and “immortalized cell line”, as used herein interchangeably, can refer to a population of cells from an organism which would normally not proliferate indefinitely but, due to mutation, may have evaded normal cellular senescence and instead can keep undergoing division. The subject matter provided herein may find use in a range of common established cell lines, including, but not limited to, human BC-1 cells, human BJAB cells, human IM-9 cells, human Jiyoye cells, human K-562 cells, human LCL cells, mouse MPC-11 cells, human Raji cells, human Ramos cells, mouse Ramos cells, human RPM18226 cells, human RS4-11 cells, human SKW6.4 cells, human Dendritic cells, mouse P815 cells, mouse RBL-2H3 cells, human HL-60 cells, human NAMALWA cells, human Macrophage cells, mouse RAW 264.7 cells, human KG-1 cells, mouse Ml cells, human PBMC cells, mouse BW5147 (T200-A) 5.2 cells, human CCRF-CEM cells, mouse EL4 cells, human Jurkat cells, human SCID.adh cells, human U-937 cells or any combination of cells thereof.

The term “primary cells” and its grammatical equivalents, as used herein, can refer to cells taken directly from an organism, typically living tissue of a multicellular organism, such as animals or plants. In many cases, primary cells may be established for growth in vitro. In some cases, primary cells may be just removed from the organism and have not been established for growth in vitro yet before the transfection. In some embodiments, the primary cells can also be expanded in vitro, i.e., primary cells may also include progeny cells that are generated from proliferation of the cells taken directly from an organism. In these cases, the progeny cells do not exhibit the indefinite proliferative property as cells in established cell lines. For instance, the host cells may be human primary T cells, while prior to the transfection, the T cells have been exposed to stimulatory factor(s) that may result in T cell proliferation and expansion of the cell population.

The cells to be genetically modified may be primary cells from tissues or organs, such as, but not limited to, brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and any combination thereof. In certain embodiments, the cells may include, but not limited to, hematocyte, trichocyte, keratinocyte, gonadotrope, corticotrope, thyrotrope, somatotrope, lactotroph, chromaffin cell, parafollicular cell, glomus cell, melanocyte, nevus cell, merkel cell, odontoblast, cementoblast, corneal keratocyte, retina muller cell, retinal pigment epithelium cell, neuron, glia, ependymocyte, pinealocyte, pneumocyte, clara cell, goblet cell, G cell, D cell, Enterochromaffin-like cell, gastric chief cell, parietal cell, foveolar cell, K cell, D cell, I cell, paneth cell, enterocyte, microfold cell, hepatocyte, hepatic stellate cell, cholecystocyte, centroacinar cell, pancreatic stellate cell, pancreatic α cell, pancreatic β cell, pancreatic δ cell, pancreatic F cell, pancreatic ε cell, thyroid parathyroid, oxyphil cell, urothelial cell, osteoblast, osteocyte, chondroblast, chondrocyte, fibroblast, fibrocyte, myoblast, myocyte, myosatellite cell, tendon cell, cardiac muscle cell, lipoblast, adipocyte, interstitial cell of cajal, angioblast, endothelial cell, mesangial cell, juxtaglomerular cell, macula densa cell, stromal cell, interstitial cell, telocyte, simple epithelial cell, podocyte, kidney proximal tubule brush border cell, sertoli cell, leydig cell, granulosa cell, peg cell, germ cell, spermatozoon ovum, lymphocyte, myeloid cell, endothelial progenitor cell, endothelial stem cell, angioblast, mesoangioblast, pericyte mural cell, and any combination thereof. In many instances, the cell to be modified may be a stem cell, such as, but not limited to, embryonic stem cell, hematopoietic stem cell, epidermal stem cell, epithelial stem cell, bronchoalveolar stem cell, mammary stem cell, mesenchymal stem cell, intestine stem cell, endothelial stem cell, neural stem cell, olfactory adult stem cell, neural crest stem cell, testicular cell, and any combination thereof. Sometimes, the cell can be an induced pluripotent stem cell that is derived from any type of tissue.

In some embodiments, the cell to be genetically modified may be a mammalian cell. In some embodiments, the cell may be an immune cell. Non-limiting examples of the cell can include a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In certain cases, the cell may be a T cell. In some embodiments, the cell may be a primary T cell. In certain cases, the cell may be an antigen-presenting cell (APC). In some embodiments, the cell may be a primary APC. The APCs in connection with the present disclosure may be a dendritic cell, macrophage, B cell, other non-professional APCs, or any combination thereof.

In some embodiments, the cell may be an ILC (innate lymphoid cell), and the ILC can be a group 1 ILC, a group 2 ILC, or a group 3 ILC. Group 1 ILCs may generally be described as cells controlled by the T-bet transcription factor, secreting type-1 cytokines such as IFN-gamma and TNF-alpha in response to intracellular pathogens. Group 2 ILCs may generally be described as cells relying on the GATA-3 and ROR-alpha transcription factors, producing type-2 cytokines in response to extracellular parasite infections. Group 3 ILCs may generally be described as cells controlled by the ROR-gamma t transcription factor, and produce IL-17 and/or IL-22.

In some embodiments, the cell may be a cell that is positive or negative for a given factor. In some embodiments, a cell may be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated cell thereof. The examples of factors expressed by cells is not intended to be limiting, and a person having skill in the art will appreciate that the cell may be positive or negative for any factor known in the art. In some embodiments, the cell may be positive for two or more factors. For example, the cell may be CD4+ and CD8+. In some embodiments, the cell may be negative for two or more factors. For example, the cell may be CD25−, CD44−, and CD69−. In some embodiments, the cell may be positive for one or more factors, and negative for one or more factors. For example, a cell may be CD4+ and CD8−.

It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells.

As provided herein, the transposase and the transposon can be introduced into a cell through a number of approaches. The term “transfection” and its grammatical equivalents as used herein can generally refer to a process whereby nucleic acids are introduced into eukaryotic cells. The transfection methods that can be used in connection with the subject matter can include, but not limited to, electroporation, microinjection, calcium phosphate precipitation, cationic polymers, dendrimers, liposome, microprojectile bombardment, fugene, direct sonic loading, cell squeezing, optical transfection, protoplast fusion, impalefection, magnetofection, nucleofection, or any combination thereof. In many cases, the transposase and transposon described herein can be transfected into a host cell through electroporation. Sometimes, transfection can also be done through a variant of electroporation method, such as nucleofection (also known as Nucleofector™ technology). The term “electroporation” and its grammatical equivalents as used herein can refer to a process whereby an electrical field is applied to cells in order to increase the permeability of the cell membrane, allowing chemicals, drugs, or DNA to be introduced into the cell. During electroporation, the electric filed is often provided in the form of “pulses” of very brief time periods, e.g., 5 milliseconds, 10 milliseconds, and 50 milliseconds. As understood by those skilled in the art, electroporation temporarily opens up pores in a cell's outer membrane by use of pulsed rotating electric fields. Methods and apparatus used for electroporation in vitro and in vivo are also well know-n. Various electric parameters can be selected dependent on the cell type being electroporated and physical characteristics of the molecules that are to be taken up by the cell, such as pulse intensity, pulse length, number of pulses).

DNA transfection can be simple and may require simply mixing naked DNA molecules together with transfection reagents, such as cation-ionic lipids, liposomes, gold or other nano-particles, or other reagents and then applying the mixture to cells. Naked DNA can also be introduced into cells by electroporation, nucleofection, biolistic delivery of particles, direct injection, sonoporation, cell squeezing, and other methods.

In some settings, special DNA sequences are placed adjacent to the introduced transgene DNA, usually flanking the transgene, which in conjunction with introduction of a special enzymes called transposases, can catalyze efficient integration of the transgene into the chromosomes of cells. These methods can enhance the efficiency of delivery of transgenes dramatically. Such methods include enhanced, permanent transgene delivery by use of DNA transposons like Sleeping Beauty. PiggyBac. Tol2, and others. In other technologies, site-specific recombinases are used to catalyze introduction of the transgene into chromosomes of cells. Examples of these include bacteriophage integrases such as PhiC31, which recognize certain mammalian or human sequences as substrates for site-specific integration. In other settings, a site-specific recombinase is used for permanent delivery of a transgene to a specific site in the genome that has been prepared for delivery of the transgene by the addition of special DNA sequences that act as a “landing pad”. These recombinase-mediated cassette exchange based methods can be efficient, but rely on prior addition of the “landing pad” sequences to the genome.

Viral transduction of transgenes into cells can be applied in many different settings in basic research and medicine. It can be used for delivery of therapeutic transgenes to primary human cells in vivo or ex vivo prior to re-infusion into a patient. For example, retroviral or lentiviral vectors can be used for human gene therapy to cure certain blood disorders, or to deliver chimeric antigen receptor (CAR) transgenes to human T cells for cancer therapy. While these methods combine efficient transgene delivery with lack of cellular toxicity, they are cumbersome, expensive, difficult to scale-up for clinical use and raise safety concerns. Therefore, methods that combine the ease of DNA transfection, with the efficiency of viral transduction, are highly desirable. Various implementations provided herein describe methods to enhance hAT family transposon mediated delivery of transgenes to human hematopoietic and immune cells.

Various specific improvements to hAT transposon vector mediated gene delivery to human hematopoietic and immune system cells are discussed herein. Together, they provide unexpectedly high gene transfer and targeting of the transposon vector to specific genomic regions. The utility of the implementations described herein are the genetic modification of these human cell types for various forms of gene therapy. This includes, but is not limited to, the following examples. One can use this system for correction of genetic disorders of hematopoiesis such as beta-thalassemia, Fanconi anemia, and others. Other genetic diseases that could be corrected include those in which genes encoding secreted factors are introduced into human B cells or other cell types. Examples include hemophilia A and B, lysosomal storage diseases, and more. Transgenes can be delivered to human T or NK cells that allow them to kill cancer cells in patients. These include chimeric antigen receptor (CAR) transgenes and T cell receptors. Systems and methods disclosed herein can be applied together with numerous methods for verifying transgene delivery and gene expression (see Green and Sambrook, Molecular Cloning: A Laboratory Manual 4^{th} Ed. For details). Applicable methods to detect the integrated transgenes and their expression include Southern blotting, polymerase chain reaction (PCR), in situ hybridization, and others. Applicable methods to detect expression of the transgene include northern blotting, reverse transcription-PCR (RT-PCR), western blotting, in situ hybridization, flow cytometry, and other approaches.

### Applications

The subject matter, e.g., the compositions (e.g., mutant SPIN transposases, fusion transposases, SPIN transposons), systems and methods, provided herein may find use in a wide range of applications relating to genome editing, in various aspects of modern life.

Under certain circumstances, advantages of the subject matter described herein may include, but not limited to, reduced costs, regulatory consideration, lower immunogenicity and less complexity. In some cases, a significant advantage of the present disclosure is the high transposition efficiency. Another advantage of the present disclosure, in many cases, is that the transposition system provided herein can be “tunable”, e.g., transposition can be designed to target select genomic region rather than random insertion.

One non-limiting example is related to create genetically modified cells for research and clinical applications. For example, as discussed above, genetically modified T cells can be created using the subject matter provided herein, which may find use in helping people fighting against a variety of diseases, such as, but not limited to, cancer and infectious disease.

One particular example includes generation of genetically modified primary leukocytes using the methods provided herein, and administering the genetically modified primary leukocytes to a patient in need thereof. The generation of genetically modified primary leukocytes can include introducing into a leukocyte a transposon and a mutant SPIN transposase or the fusion transposase as described herein, which can recognize the transposon, thereby generating a genetically modified leukocyte. In many cases, the transposon may comprise a transgene. The transgene can be a cellular receptor, an immunological checkpoint protein, a cytokine, and any combination thereof. Sometimes, a cellular receptor can include, but not limited to a T cell receptor (TCR), a B cell receptor (BCR), a chimeric antigen receptor (CAR), or any combination thereof. In some other cases, the transposon and the transposase are designed to delete or modify an endogenous gene, for instance, a cytokine, an immunological checkpoint protein, an oncogene, or any combination thereof. The genetic modification of the primary leukocytes can be designed to facilitate immunity against an infectious pathogen or cancer cells that render the patient in diseased state.

Another non-limiting example is related to create genetically modified organisms for agriculture, food production, medicine, and pharmaceutics. The species that can be genetically modified span a wide range, including, but not limited to, plants and animals. The genetically modified organisms, such as genetically modified crops or livestock, may be modified in a certain aspect of their physiological properties. Examples in food crops include resistance to certain pests, diseases, or environmental conditions, reduction of spoilage, or resistance to chemical treatments (e.g., resistance to a herbicide), or improving the nutrient profile of the crop. Examples in non-food crops include production of pharmaceutical agents, biofuels, and other industrially useful goods, as well as for bioremediation. Examples in livestock include resistance to certain parasites, production of certain nutrition elements, increase in growth rate, and increase in milk production.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

### EXAMPLES

The examples below further illustrate the described embodiments without limiting the scope of this disclosure.

### Example 1. Materials and Methods

This example describes several methods utilized for generation and evaluation of exemplary mutant SPIN transposases.

### Site Directed Mutagenesis for SPIN Mutant Preparation

Putative hyperactive SPIN transposase mutants were identified by nucleotide sequence and amino acid alignment of hAT and buster subfamilies. The Q5 site-directed mutagenesis kit (New England BioLabs) was used for all site-directed mutagenesis. Following PCR mutagenesis, PCR products were purified with GeneJET PCR purification kit (Thermo Fisher Scientific). A 20 uL ligation reaction of purified PCR products was performed using T4 DNA ligase (New England BioLabs). 5 uL of ligation reaction was used for transformation in DHIOBeta cells. Direct colony sequencing through Sequetech was used to confirm the presence of desired mutations. DNA for confirmed mutations was prepped using ZymoPURE plasmid miniprep kits (Zymo Research).

### Measuring Transection Efficiency in HEK-293T Cells

HEK-293T cells were plated at 300,000 cells per well of a 6 well plate one day prior to transfection. Cells were transfected with 500 ng transposon carrying mCherry-puromycin cassette and 62.5 ng SPIN transposase using TransIT X2 reagent per manufacturer's instructions (Mirus Bio). Two days post-transfection, cells were re-plated with puromycin (1 ug/mL) at a density of 3,000 cells/well of a 6 well plate in triplicate in DMEM complete media, or re-plated without puromycin selection. Stable integration of the transgene was assessed by colony counting of puromycin treated cells (each cell that survived drug selection formed a colony) or flow cytometry. For colony counting, two weeks post-puromycin selection, DMEM complete+puromycin media was removed. Cells were washed with 1×PBS and cells were stained with 1×crystal violet solution for 10 minutes. Plates were washed twice with PBS and colonies counted.

For flow cytometry analysis, stable integration of the transgene was assessed by detection of mCherry fluorescence in cells grown without drug selection. Transfected cells were harvested at indicated time points post-transfection, washed 1× with PBS and resuspended in 200 uL RDFII buffer for analysis. Cells were analyzed using Novocyte (Acea Biosciences) and mCherry expression was assessed using the PE-Texas red channel.

### Screening of SPIN Transposase Mutants in HEK-293T Cells

HEK-293T cells were plated at 75,000 cells per well of a 24 well plate one day prior to transfection. Cells were transfected with 500 ng transposon and 125 ng transposase using TransIT X2 reagent in duplicate per manufacturer's instructions (Mirus Bio). Stable integration of the transgene was assessed by detection of mCherry fluorescence. Cells were harvested at 14 days post-transfection, washed IX with PBS and resuspended in 200 uL RDFII buffer. Cells were analyzed using Novocyte (Acea Biosciences) and mCherry expression was assessed using the PE-Texas red channel.

### Example 2. Exemplary Transposase Mutants

The aim of this study was to generate SPIN transposase mutants and examine their transposition efficiency.

To this end, inventors have generated a consensus sequence by comparing cDNA and amino acid sequences of wild-type SPIN transposase to other similar transposases. For the comparison, sleeping beauty was resurrected by the alignment of 13 similar transposases and SPIN by the alignment of SPIN like transposases from 8 separate organisms. SPIN is a part of the abundant hAT family of transposases.

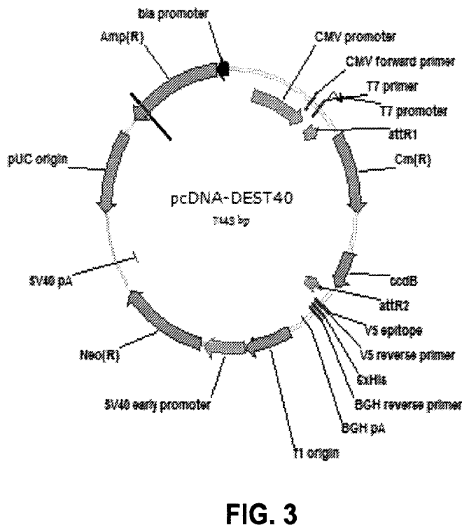

The hAT transposon family consists of two subfamilies: AC, such as has hobo, hermes, and Tol2, and the Buster subfamily, such as SPIN and TcBuster. Amino acid sequence of SPIN was aligned to amino acid sequences of the Buster subfamily members to identify key amino acids that are not conserved in SPIN that may be targets of hyperactive substitutions. Sequence alignment of SPIN to the Buster subfamily led to a larger number of candidate amino acids that may be substituted (FIG. 2). The mutants were then sequence verified, cloned into pCDNA-DEST40 expression vector (FIG. 3) and mini-prepped prior to transfection.

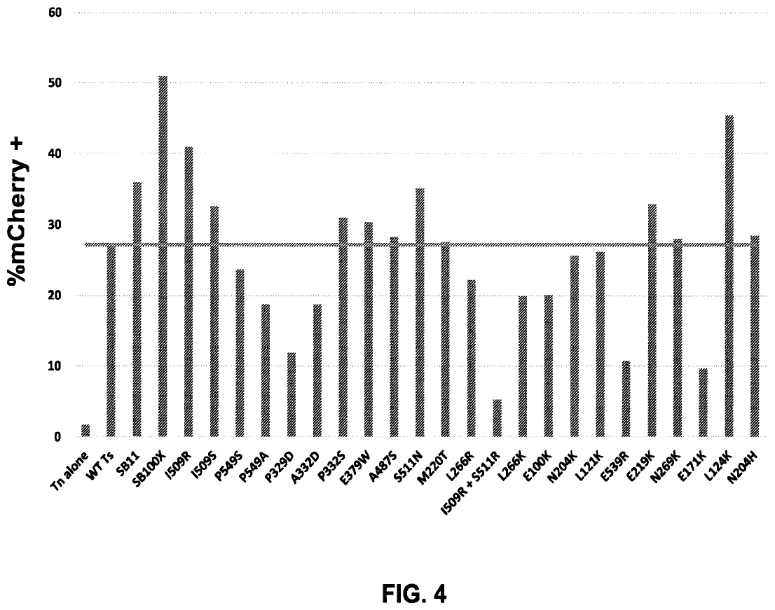

To examine the transposition efficiency of the SPIN transposase mutants, HEK-293T cells were transfected with SPIN Tn (mCherry-puromycin cassette) with WT transposase or V596A mutant transposase, or the candidate transposase mutants in duplicate. Cells were grown in DMEM complete (without drug selection) and mCherry expression was assessed by flow cytometry on Day 14 post-transfection. 5 SPIN transposase mutants were identified that had transposition efficiency greater than the wild-type transposase (FIG. 4). It was discovered that among these examined mutants, one mutant transposase containing a combination of two amino acid substitutions, I509R and L124K led to a substantial increase in transposition activity, as compared to mutants containing respective single substitutions.

Among these examined mutants, it was discovered that most of substitutions to a positively charged amino acid, such as Lysine (K) or Arginine (R), in proximity to one of the catalytic triad amino acids (D185, D251, and E555) increased transposition. These data suggest that amino acids close to the catalytic domain may help promote the transposition activity of SPIN, in particular, when these amino acids are mutated to positively charged amino acids.

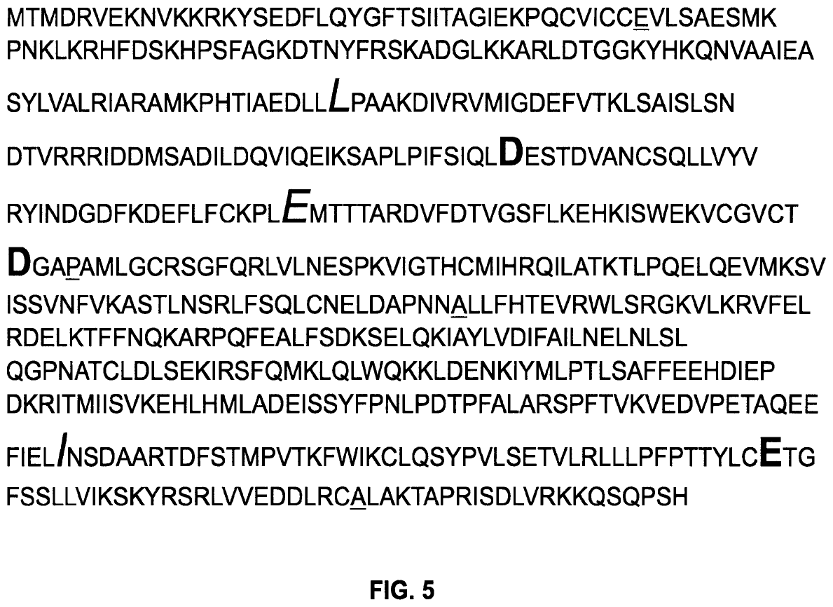

FIG. 5 depicts the WT SPIN transposase amino acid sequence, in which large bold underlined lettering indicates catalytic triad amino acids: large italicized lettering indicates amino acids that when substituted to a positive charged amino acid increase transposition; underlined lettering indicates amino acids that could be positive charged amino acids based on protein sequence alignment to the Buster subfamily.

### Example 3. Exemplary Fusion Transposase Containing Tag

The aim of this study is to generate and examine the transposition efficiency of fusion SPIN transposases. As an example, protein tag, GST or PEST domain, is fused to N-terminus of SPIN transposase to generate fusion SPIN transposases. A flexible linker GGSGGSGGSGGSGTS (SEQ ID NO: 7), which is encoded by SEQ ID NO: 8, is used to separate the GST domain/PEST domain from SPIN transposase. The presence of this flexibility linker may minimize non-specific interaction in the fusion protein, thus increasing its activity. The exemplary fusion transposases are transfected with SPIN Tn as described above and transposition efficiency is measured by mCherry expression on Day 14 by flow cytometry.

### Example 4. Exemplary Fusion Transposase Comprising Tale Domain

The aim of this study is to generate a fusion SPIN transposase comprising a TALE domain and to examine the transposition activity of the fusion transposase. A TALE sequence SEQ ID NO: 9 is designed to target human AAVS1 (hAAVS1) site of human genome. The TALE sequence is thus fused to N-terminus of a wild-type SPIN transposase (SEQ ID NO: 1) to generate a fusion transposase. A flexible linker Gly4Ser2, which is encoded by SEQ ID NO: 10, is used to separate the TALE domain and the SPIN transposase sequence. The exemplary fusion transposase has an amino acid sequence SEQ ID NO: 6.

The exemplary fusion transposase will be transfected with a SPIN Tn as described above into Hela cells with the aid of electroporation. The SPIN Tn comprises a reporter gene mCherry. The transfection efficiency can be examined by flow cytometry 2 days post-transfection that counts mCherry-positive cells. Furthermore, next-generation sequencing will be performed to assess the mCherry gene insertion site in the genome. It is expected that the designed TALE sequence can mediate the target insertion of the mCherry gene at a genomic site near hAAVS1 site.

### Example 5. Generation of Chimeric Antigen Receptor-Modified T Cells for Treatment of Cancer Patient

A mini-circle plasmid containing aforementioned SPIN Tn construct can be designed to harbor a chimeric antigen receptor (CAR) gene between the inverted repeats of the transposon. The CAR can be designed to have specificity for the B-cell antigen CD19, coupled with CD137 (a costimulatory receptor in T cells [4-1BB]) and CD3-zeta (a signal-transduction component of the T-cell antigen receptor) signaling domains.

Autologous T cells will be obtained from peripheral blood of a patient with cancer, for example, leukemia. The T cells can be isolated by lysing the red blood cells and depleting the monocytes by centrifugation through a PERCOLL™ gradient. CD3+ T cells can be isolated by flow cytometry using anti-CD3/anti-CD28-conjugated beads, such as DYNABEAD M-450 CD3/CD28T. The isolated T cells will be cultured under standard conditions according to GMP guidance.

Genetic modification of the primary T cells will be conducted using a mutant SPIN transposase (SEQ ID NO: 11) comprising amino acid substitutions I509R and L124K and the SPIN Tn (transposon) comprising the CAR, as described above. The T cells will be electroporated in the presence of the mutant SPIN transposase and the CAR-containing SPIN Tn. Following transfection, T cells will be treated with immunostimulatory reagents (such as anti-CD3 antibody and IL-2, IL-7, and IL-15) for activation and expansion. Validation of the transfection will be performed by next-generation sequencing 2 weeks post-transfection. The transfection efficiency and transgene load in the transfected T cells can be determined to assist the design of treatment regimen. Certain measure will also be taken to eliminate any safety concern if risky transgene insertion site is uncovered by the sequencing results.

Infusion of the chimeric antigen receptor modified T cells (CAR-T cells) back to the cancer patient will start after validation of transgene insertion and in vitro expansion of the CAR-T cells to a clinically desirable level.

The infusion dose will be determined by a number of factors, including, but not limited to, the stage of the cancer, the treatment history of the patient, and the CBC (complete blood cell count) and vital signs of the patient on the day of treatment. Infusion dose may be escalated or deescalated depending on the progression of the disease, the repulsion reaction of the patient, and many other medical factors. In the meantime, during the treatment regimen, quantitative polymerase-chain-reaction (qPCR) analysis will be performed to detect chimeric antigen receptor T cells in blood and bone marrow. The qPCR analysis can be utilized to make medical decision regarding the dosing strategy and other treatment plans.

### Example 6. Dual Transposase for Virus Production

A dual transposase system may be used for the creation of a producer cell line for viral production. This may be achieved by the stable introduction of viral packaging and viral production helper genes with a first transposase system, and subsequent stable introduction of a gene of interest within the respective viral ITRs/LTRs by a second transposase system. This system results in an ideal ratio of stably expressed helper genes, packaging genes, and gene(s) of interest, yielding viruses of high titer and quality without the remobilization and potential loss of genes that would occur if just one transposase system was used.

The dual transposase system may be used for stable integration of viral packaging genes to product lentivirus, such as gagpol, rev, and VsVg. The dual transposase system also may be used to generate cells for adeno-associated virus (AAV) production. For example, a dual transposase system may be used to stably integrate helper genes into cells, such as, for example, adenovirus helper genes (e.g., E1B19K, E1B55K, protein IX, E4orf6, E2A, E1B55K/E4orf6, E1A, and VA-RNA), HSV-1 helper genes (e.g., UL5, UL8, UL52, ICP8, HP, UL30, UL42, ICP0, ICP4, and ICP22); HPV-16 helper genes (e.g., E2, E1, E6); and HBoV1 helper genes (e.g., NS2, NS4, NP1, BocaSR). Genes for AAV packaging also may be stably integrated into cells using the dual transposase system. Such packaging genes include, but are not limited to, AAV rep and cap genes, and adenovirus E1, E2, E3, and E4 genes.

### Example 7. Dual Transposase for Recombinant Protein and Monoclonal Antibody Production

A dual transposase system may be used to produce recombinant proteins and monoclonal antibodies. For example, a first transposase may be employed to stably express genes encoding glycosyltransferases and hydrolases (which function in N-glycosylation and M6P processing) and/or sialyltransferases (which promote cell viability and increased productivity). A second transposase may then be used to stably introduce and express a gene encoding a recombinant protein or monoclonal antibody of interest. Other genes involved in posttranslational modification which may be integrated into a cellular genome using the dual transposase system described herein include, for example. Mgat3, sialylation enzyme genes (e.g., st6gal1 (α2,6-sialyltransferase 1), ST3GAL4 (α2,3-sialyltransferase 4), D1,4-galactosyltransferase 1, CMP-sialic acid synthase. UDP-GlcNAc 2-epimerase/ManNAc kinase, α-1,3-d-mannoside β-1,4-Nacetylglucosaminyltransferase α-1,6-d-mannoside D-1,6-Nacetylglucosaminyltransferase. N-acetylglucosaminyltransferase I, an anti-apoptotic member of the Bcl-2 family, 30Kc19 cell penetrating peptide), human D1,4-galactosyltransferase (β1,4-GalT), α2,3-sialyltransferase (α2,3-ST), GnT-IV, GnT-V, and endo-b-N-acetylglucosaminidases. Genes that increase recombinant protein production which may be integrated into a cellular genome using the dual transposase system described herein include, for example, mTOR signaling promoters, transcription factors (e.g., ATF4), and CHOP/Gadd153 and GRP78 (which are unfolded protein response (UPR)-related genes). The dual transposase system described herein may be used to stably integrate into a cellular genome genes involved in increasing cell viability, such as, e.g., the antiapoptotic genes Bcl-2, Bel-x, and Mcl-1.

TABLE 7
Amino Acid and Nucleotide Sequences

| Sequence | SEQ | |
|---|---|---|
| Description | ID NO: | Amino Acid Sequence or Nucleotide Sequence |
| Wild-type | 1 | (accession number: ABF20545) |
| SPIN | | MIMDRVEKNVKKRKYSEDFLQYGFTSIITAGIERPQCVICCEVLSAESMKPNKLKRHFDSKHPSFAGKD |
| transposase | | TNYFRSKADGLKKARLDTGGKYHKONVAAIEASYLVALRIARAMKPHTIAEDLLLPAAKDIVRVMIGDE |
| | | FVTKLSAISLSNDTVRRRIDDMSADILDOVIQEIKSAPLPIFSIQLDESTDVANCSQLLVYVRYINDGD |
| | | FKDEFLFCKPLEMTTTARDVFDTVGSFLKEHKISWEKVCGVCTDGAPAMLGCRSGFORLVLNESPKVIG |
| | | THCMIHRQILATKTLPQELQEVMKSVISSVNFVKASTLNSRLFSQLCNELDAPNNALLFHTEVRWLSRG |
| | | KVLKRVFELRDELKTFFNQKARPQFEALFSDKSELQKIAYLVDIFAILNELNLSLOGPNATCLDLSEKI |
| | | RSFQMKLQLWQKKLDENKIYMLPTLSAFFEEHDIEPDKRITMIISVKEHLHMLADEISSYFPNLPDTPF |
| | | ALARSPFTVKVEDVPETAQEEFIELINSDAARTDFSTMPVTKFWIKCLQSYPVLSETVLRLLLPFPTTY |
| | | LCETGFSSLLVIKSKYRSRLVVEDDLRCALAKTAPRISDLVRKKQSQPSH |
| Wild-type | 2 | atgaccatggaccgcgttgaaaagaatgtaaaaaagagaaagtatagtgaggatttcttgcagtatgga |
| SPIN | | tttacttccattatcactgcgggtattgaaaaacctcaatgtgttatatgttgcgaagtcctgtcagca |
| transposase | | gaatcaatgaaacctaataaattgaaaaggcatttcgattccaagcatccaagcttcgctgggaaggat |
| | | accaactattttaggtctaaagctgacggtcttaaaaaagcgcggttggatacaggtggtaagtaccac |
| | | aagcagaatgtggcggctatcgaggcgtcctatctggttgcacttcgcatcgctagagctatgaaacca |
| | | cacaccatcgcagagcgatatcactctcaaacgacacggtacgacggaggatagatgacatgagtgctg |
| | | acatattggaccaggtgatacaggaaattaagtctgctccccttccgatattctctattcaactcgacg |
| | | aaagcaccgatgttgcaaattgttcacagttgttggtatatgtacggtatattaatgatggggatttca |
| | | aagatgagttcctgttttgtaagcctcttgaaatgaccaccacagcccgggatgtattcgacactgtcg |
| | | gtagcttccttaaagaacacaaaattagctgggaaaaggtctgtggtgtttgtacggacggtgctccgg |
| | | cgatgctgggatgcagatcaggatttcaaagactcgtgcttaacgagtctcctaaggtgataggcactc |
| | | actgtatgatacaccggcaaattctcgcaaccaagacattgccacaggaacttcaagaagttatgaagt |
| | | ctgtaatatcatccgtaaatttcgtgaaagcaagtactctgaactcacgactcttttcacaactttgta |
| | | atgagcttgacgcacccaacaacgccctgttgtttcatacagaagtccggtggctgagtcgcgggaaag |
| | | tacttaagagggtattcgagctccgggacgagctgaagacatttttcaaccagaaggcacgaccccaat |
| | | ttgaggcgctgtttagcgataagagcgaacttcagaagatcgcgtaccttgtggatatcttcgcaattt |
| | | tgaacgaactcaacttgtctctgcaagggcctaatgccacgtgcctggacctttccgagaagattagat |
| | | ccttccagatgaagttgcagctgtggcagaaaaagctggatgaaaacaagatttatatgttgccgacac |
| | | tttccgcatttttcgaggaacacgacattgaaccagacaaacgcatcacaatgattatctcagtgaaag |
| | | agcacttgcacatgttggccgacgaaatttcatcctattttccaaatcttccagatactccgttcgctc |
| | | tcgcacgcagccctttcacggtaaaagttgaagacgtaccagaaacggcacaggaggagttcattgaac |
| | | tgattaattctgatgctgcccgcactgacttttccacgatgccagttacgaaattttggattaaatgtc |
| | | ttcagtcctatcccgttcttagtgagacggtattgcggcttcttctcccatttccgaccacgtacctct |
| | | gtgaaacgggattctcatccttgctggtgatcaaaagcaagtaccgatcccgactcgtggtcgaagatg |
| | | accttcgatgcgccctcgcaaaaactgcaccccggatcagcgacttggtgagaaagaaacaatctcaac |
| | | caagtcactga |
| L-ITR-Seq | 3 | cagcggttctcaacctgtgggtcgcgacccctttgggggtcaaacgaccctttcacaggggtcgcctaa |
| | | gaccatcggaaaacacatatttccgatggtcttaggaaccgagacaccgctcctctatccgtctccagg |
| | | cgggtccgcccacatgcagatacgcccacataggagtacccggcgtgatgacatcatcgcgccaacccc |
| | | atcacatacaccccgtacaaatacaggtgtatgtgacagggttggcgccataatgtacttatgcggacc |
| | | agtcacacatgtgtagagagcagctactgtgtagaaagcagctactgtgttgaaagcagctactctgtt |
| | | aaaagcagctactgtgttgaaagcagcagtattggaggtaaaacgacacttcatgaattataattactg |
| | | ggtaaatgtaaaattcatgtactgtaaaatcatcaactactgcaaaaaaaaaaaaatatctgtaccatg |
| | | gggaacttaatctggatgctgatcggtctttttatattcagctgtggttgatgtgaatactgccccctt |
| | | gtgatagtaacaggtatgtaaaaaaaaacaacacagagaatggtaaatcataggaaactttaatgaact |
| | | gtattgactgaactatgccatgtatcatcttttgtattattaaagctattgttatatattattttcatt |
| | | agcaaaccatccc |
| R-ITR-Seg | 4 | cgttggctttttacgcatactgtcgcaaaatgtagcaatgtagtttactgttgttatattaagactgtt |
| | | acccatgctacaccatgcttcaagacaaaatttcatttatttgtaattagaaataaatatttcacaata |
| | | tataattacatattgtttttgtgattaatcactatgctttaattatgttcaatttgtaacaatgaaaat |
| | | acatcctgcatatcagatatttacattacgattcataacagtagcaaaattacagitatgaagtagcaa |
| | | cgaaaataattttatggttgggggtcaccacaacatgaggaactgtattaaagggtcgcggcattagga |
| | | aggttgagaaccactg |
| pcDNA-DEST | 5 | gacggatcgggagatctcccgatcccctatggtgcactctcagtacaatctgctctgatgccgcatagt |
| 40 | | taagccagtatctgctccctgcttgtgtgttggaggtcgctgagtagtgcgcgagcaaaatttaagcta |
| | | caacaaggcaaggcttgaccgacaattgcatgaagaatctgcttagggttaggcgttttgcgctgcttc |
| | | gcgatgtacgggccagatatacgcgttgacattgattattgactagttattaatagtaatcaattacgg |
| | | ggtcattagttcatagcccatatatggagttccgcgttacataacttacggtaaatggcccgcctggct |
| | | gaccgcccaacgacccccgcccattgacgtcaataatgacgtatgttcccatagtaacgccaataggga |
| | | ctttccattgacgtcaatgggtggagtatttacggtaaactgcccacttggcagtacatcaagtgtatc |
| | | atatgccaagtacgccccctattgacgtcaatgacggtaaatggcccgcctggcattatgcccagtaca |
| | | tgaccttatgggactttcctacttggcagtacatctacgtattagtcatcgctattaccatggtgatgc |
| | | ggttttggcagtacatcaatgggcgtggatagcggtttgactcacggggatttccaagtctccacccca |
| | | ttgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaa |
| | | ctagagaacccactgcttactggcttatcgaaattaatacgactcactatagggagacccaagctggct |
| | | agttaagctatcaacaagtttgtacaaaaaagctgaacgagaaacgtaaaatgatataaatatcaatat |
| | | attaaattagatttigcataaaaaacagactacataatactgtaaaacacggatataccaccgttgata |
| | | tatcccaatggcatcgtaaagaacattttgaggcatttcagtcagttgctcaatgtacctataaccaga |
| | | ccgttcagctggatattacggcctttttaaagaccgtaaagaaaaataagcacaagttttatccggcct |
| | | ttattcacattcttgcccgcctgatgaatgctcatccggaattccgtatggcaatgaaagacggtgagc |
| | | tggtgatatgggatagtgttcacccttgttacaccgttttccatgagcaaactgaaacgttttcatcgc |
| | | tctggagtgaataccacgacgatttccggcagtttctacacatatattcgcaagatgtggcgtgttacg |
| | | gtgaaaacctggcctatttccctaaagggtttattgagaatatgtttttcgtctcagccaatccctggg |
| | | tgagtttcaccagttttgatttaaacgtggccaatatggacaacttcttcgcccccgttttcaccatgg |
| | | gcaaatattatacgcaaggcgacaaggtgctgatgccgctggcgattcaggttcatcatgccgtctgtg |
| | | atggcttccatgtcggacccgaagtatgtcaaaaagaggtgtgctatgaagcagcgtattacagtgaca |
| | | gttgacagcgacagctatcagttgctcaaggcatatatgatgtcaatatctccggtctggtaagcacaa |
| | | ccatgcagaatgaagcccgtcgtctgcgtgccgaacgctggaaagcggaaaatcaggaagggatggctg |
| | | aggtcgcccggtttattgaaatgaacggctcttttgctgacgagaacagggactggtgaaatgcagttt |
| | | aaggtttacacctataaaagagagagccgttatcgtctgtttgtggatgtacagagtgatattattgac |
| | | acgcccgggcgacggatggtgatggtgatccccctggccagtgcacgtctgctgtcagataaagtctcc |
| | | cgtgaactttacccggtggtgcatatcggggatgaaagctggcgcatgatgaccaccgatatggccagt |
| | | gtgccggtctccgttatcggggggggaagaagtggctgatctcagccaccgcgaaaatgacatcaaaaa |
| | | cgccattaacctgatgttctggggaatataaatgtcaggctccgttatacacagccagtctgcaggtcg |
| | | accatagtgactggatatgttgtgttttacagtattatgtagtctgttttttatgcaaaatctaattta |
| | | atatattgatatttatatcattttacgtttctcgttcagctttcttgtacaaagtggttgatctagagg |
| | | gcccgcggttcgaaggtaagcctatccctaaccctctcctcggtctcgattctacgcgtaccggtcatc |
| | | atcaccatcaccattgagtttaaacccgctgatcagcctcgactgtgccttctagttgccagccatctg |
| | | ttgtttgcccctcccccgtgccttccttgaccctggaaggtgccactcccactgtcctttcctaataaa |
| | | atgaggaaattgcatcgcattgtctgagtaggtgtcattctattctggggggtggggtggggcaggaca |
| | | gcaagggggaggattgggaagacaatagcaggcatgctggggatgcggtgggctctatggcttctgagg |
| | | cggaaagaaccagctggggctctagggggtatccccacgcgccctgtagcggcgcattaagcgcggcgg |
| | | gtgtggtgtttttcgccctttgacgttggagtccacgttctttaatagtggactcttgttccaaactgg |
| | | aacaacactcaaccaaaaatttaacgcgaattaattctgtggaatgtgtgtcagttagggtgtggaaag |
| | | tccccaggctccccagcaggcagaagtatgcaaagcatgcatctcaattagtcagcaaccaggtgtgga |
| | | aagtccccaggctccccagcaggcagaagtatgcaaagcatgcatctcaattagtcagcaaccatagtc |
| | | ccgcccctaactccgcccatcccgcccctaactccgcccagttccgcccattctccgccccatggctga |
| | | ctaattttttttatttatgcagagaaaaagctcccgggagcttgtatatccattttcggatctgatcaa |
| | | gagacaggatgaggatcgtttcgcatgattgaacaagatggattgcacgcaggttctccggccgcttgg |
| | | gtggagaggctattcggctatgactgggctcaagaccgacctgtccggtgccctgaatgaactgcagga |
| | | cgaggcagcgcggctatcgtggctggccacgacgggcgttccttgcgcagctgtgctcgacgttgtcac |
| | | tgaagcgggaagggactggctgctattgggcgaagtgccggggcaggatctcctgtcatctcaccttgc |
| | | tcctgccgagaaagtatccatcatggctgatggcgagcacgtactcggatggaagccggtcttgtcgat |
| | | caggatgatctggacgaagagcatcaggggctcgcgccagccgaactgttcgccaggctcaaggcgcgc |
| | | atgcccgacggcgaggatctcgtcgtgacccatggcgatgcctgcttgccgaatatcatggtggaaaat |
| | | ggccgcttttctggattcatcgactgtggccggctggggggggcggaccgctatcaggacatagcgttg |
| | | gctacccgtgatattgctgaagagcttggcggcgaatgacgagttcttctgagcgggactctggggttc |
| | | gcgaaatgaccgaccaagcgacgcccaacctgccatcgitacaaataaagcaatagcatcacaaatttc |
| | | acaaataaagcatttttttcactgcattctagttgtggtttgtccaaactcatcaatgtatcttatcat |
| | | gtctgtataccgtcgacctctagctagagcttggcgtaatcatggtcatagctgtttcctgtgtgaaat |
| | | tgttatccgctcacaattccacacaacatacgagccggaagcataaagtgtaaagcctggggtgcctaa |
| | | tgagtgagctaactcacattaattgcgttgcgctcactgcccgctttccagtcgggaaacctgtcgtgc |
| | | cagctgcattaatgaatcggccaacgcgcggggagaggcggtttgcgtattgggcgctaggcggtaata |
| | | cggttatccacagaatcaggggataacgcaggaaagaacatgtgagcaaaaggccagcaaaaggccagg |
| | | aaccgtaaaaaggccgcgttgctggcgtttttccataggctccgcccccctgacgagcatcacaaaaat |
| | | cgacgctcaagtcagaggtggcgaaacccgacaggactataaagataccaggcgtttccccctggaagc |
| | | tccctcgtgcgctctcctgttccgaccctgccgcttaccggatacctgtccgcctttctcccttcggga |
| | | agcgtggcgctttctcatagctcacgctgtaggtatctcagttcggtgtaggtcgttcgctccaagctg |
| | | ggctgtgtgcacgaaccccccgttcagcccgaccgctgcgccttatccggtaactatcgtcttgagtcc |
| | | aacccggtaagacacgacttatcgccactggcagcagccactggtaacaggattagcagagcgaggtat |
| | | gtaggcggtgctacagagttcttgaagtggtggcctaactacggctacactagaagaacagtatttggt |
| | | atctggtagcggtggtttttttgtttgcaagcagcagattacgcgcagaaaaaaaggatctcaagaaga |
| | | tcctttgatcttttctacggggtctgacgctcagtggaacgaaaactcacgttaagggattttggtcat |
| | | gagattatcaagtctgacagttaccaatgcttaatcagtgaggcacctatctcagcgatctgtctattt |
| | | cgttcatccatagttgcctgactccccgtcgtgtagataactacgatacgggagggcttaccatctggc |
| | | cccagtgctgcaatgataccgcgagacccacgctcaccggctccagatttatcagcaataaaccagcca |
| | | gccggaagggccgagcgcagaagtggtcctgcaactttatccgcctccatccagtctattaattgttgc |
| | | cgggaagctagagtaagtagttcgccagttaatagtttgcgcaacgttgttgccattgctacaggcatc |
| | | gtggtgtcacgctcgtcgtttggtatggcttcattcagctccggttcccaacgatcaaggcgagttaca |
| | | tgatcccccatgttgtgcaaaaaagcggttagcataattctcttactgtcatgccatccgtaagatgct |
| | | tttctgtgactggtgagtactcaaccaagtcattctgagaactttaaaagtgctcatcattggaaaacg |
| | | ttcttcggggcgaaaactctcaaggatcttaccgctgttgagagcaaaaacaggaaggcaaaatgccgc |
| | | aaaaaagggaataagggcgacacggaaatgttgaatactcatactcttcctttttcaatattattgaag |
| | | catttatcagggttattgtctcatgagcggatacatatttgaatgtatttagaaaaataaacaaatagg |
| | | ggttccgcgcacatttccccgaaaagtgccacctgacgtc |
| Fusion | 6 | atgctcgagatggatccctccgacgcttcgccggccgcgcaggtggatctacgcacgctcggctacagt |
| Transposase | | cagcagcagcaagagaagatcaaaccgaaggtgcgttcgacagtggcgcagcaccacgaggcactggtg |
| containing | | ggccatgggtttacacacgcgcacatcgttgcgctcagccaacacccggcagcgttagggaccgtcgct |
| wild-type SPIN | | gtcacgtatcagcacataatcacggcgttgccagaggcgacacacgaagacatcgttggcgtcggcaaa |
| sequence and | | cagtggtccggcgcacgcgccctggaggccttgttgactgatgctggtgagcttagaggacctcctttg |
| TALE DNA- | | caacttgatacaggccagcttctgaaaatcgccaagaggggtggggtcaccgcggtcgaggccgtacac |
| binding domain | | gcctggagaaatgcactgaccggggctcctcttaacCTGACCCCAGACCAGGTAGTCGCAATCGCGTCA |
| targeting | | AACGGAGGGGGAAAGCAAGCCCTGGAAACCGTGCAAAGGTTGTTGCCGGTCCTTTGTCAAGACCACGGC |
| human AAVS1 | | CTTACACCGGAGCAAGTCGTGGCCATTGCATCCCACGACGGTGGCAAACAGGCTCTTGAGACGGTTCAG |
| (TALE domain | | AGACTTCTCCCAGTTCTCTGTCAAGCCCACGGGCTGACTCCCGATCAAGTTGTAGCGATTGCGTCGCAT |
| italicized, | | GACGGAGGGAAACAAGCATTGGAGACTGTCCAACGGCTCCTTCCCGTGTTGTGTCAAGCCCACGGTTTG |
| Gly4Ser2 | | ACGCCTGCACAAGTGGTCGCCATCGCCTCCAATATTGGCGGTAAGCAGGCGCTGGAAACAGTACAGCGC |
| linker | | CTGCTGCCTGTACTGTGCCAGGATCATGGACTGACGGCCAAGCTGGCCGGGGGCGCCCCCGCCGTGGGC |
| underlined, and | | GGGGGCCCCAAGGCCGCCGATAAATTCGCCGCCACCatgaccatggaccgcgttgaaaagaatgtaaaa |
| SPIN | | aagagaaagtatagtgaggatttcttgcagtatggatttacttccattatcactgcgggtattgaaaaa |
| transposase | | cctcaatgtgttatatgttgcgaagtcctgtcagcagaatcaatgaaacctaataaattgaaaaggcat |
| sequence bold) | | ttcgattccaagcatccaagcttcgctgggaaggataccaactattttaggtctaaagctgacggtctt |
| | | aaaaaagcgcggttggatacaggtggtaagtaccacaagcagaatgtggcggctatcgaggcgtcctat |
| | | ctggttgcactttogagtgatgataggagatgaattcgtaacgaaactttctgcgatatcactctcaaa |
| | | cgacacggtacgacggaggatagatgacatgagtgctgacatattggaccaggtgatacaggaaattaa |
| | | gtctgctccccttccgatattctctattcaactcgacgaaagcaccgatgttgcaaattgttcacagtt |
| | | gttggtatatgtacggtatattaatgatggggatttcaaagatgagttcctgttttgtaagcctcttga |
| | | aatgaccaccacagcccgggatgtattcgacactgtcggtagcttccttaaagaacacaaaattagctg |
| | | ggaaaaggtctgtggtgtttgtacggacggtgctccggcgatgctgggatgcagatcaggatttcaaag |
| | | actcgtgcttaacgagtctcctaaggtgataggcactcactgtatgatacaccggcaaattctcgcaac |
| | | caagacattgccacaggaacttcaagaagttatgaagtctgtaatatcatcogtaaatttcgtgaaagc |
| | | aagtactctgaactcacgactcttttcacaactttgtaatgagcttgacgcacccaacaacgccctgtt |
| | | gtttcatacagaagtccggtggctgagtcgcgggaaagtacttaagagggtattcgagctccgggacga |
| | | gctgaagacatttttcaaccagaaggcacgaccccaatttgagtcaacttgtctctgcaagggcctaat |
| | | gccacgtgcctggacctttccgagaagattagatccttccagatgaagttgcagctgtggcagaaaaag |
| | | ctggatgaaaacaagatttatatgttgccgacactttccgcatttttcgaggaacacgacattgaacca |
| | | gacaaacgcatcacaatgattatctcagtgaaagagcacttgcacatgttggccgacgaaatttcatcc |
| | | tattttccaaatcttccagatactccgttcgctctcgcacgcagccctttcacggtaaaagttgaagac |
| | | gtaccagaaacggcacaggaggagttcattgaactgattaattctgatgctgcccgcactgacttttcc |
| | | acgatgccagttacgaaattttggattaaatgtcttcagtcctatcccgttcttagtgagacggtattg |
| | | cggcttcttctcccatttccgaccacgtacctctgtgaaacgggattctcatccttgctggtgatcaaa |
| | | agcaagtaccgatcccgactcgtggtcgaagatgaccttogatgcgccctcgcaaaaactgcaccccgg |
| | | atcagcgacttggtgagaaagaaacaatctcaaccaagtcactga |
| Flexible linker | 7 | GGSGGSGGSGGSGTS |
| (Example 4) | | |
| Flexible linker | 8 | GGAGGTAGTGGCGGTAGTGGGGGCTCCGGIGGGAGCGGCACCTCA |
| (Example 4) | | |
| TALE domain | 9 | atgctcgagatggatccctccgacgcttcgccggccgcgcaggtggatctacgcacgctcggctacagt |
| targeting | | cagcagcagcaagagaagatcaaaccgaaggtgcgttcgacagtggcgcagcaccacgaggcactggtg |
| hAAVS1 site | | ggccatgggtttacacacgcgcacatcgttgcgctcagccaacacccggcagcgttagggaccgtcgct |
| (Example 5) | | gtcacgtatcagcacataatcacggcgttgccagaggcgacacacgaagacatcgttggcgtcggcaaa |
| | | cagtggtccggcgcacgcgccctggaggccttgttgactgatgctggtgagcttagaggacctcctttg |
| | | caacttgatacaggccagcttctgaaaatcgccaagaggggtggggtcaccgcggtcgaggccgtacac |
| | | gcctggagaaatgcactgaccggggctcctcttaacCTGACCCCAGACCAGGTAGTCGCAATCGCGTCA |
| | | AACGGAGGGGGAAAGCAAGCCCTGGAAACCGTGCAAAGGTTGTTGCCGGTCCTTTGTCAAGACCACGGC |
| | | CTTACACCGGAGCAAGTCGTGGCCATTGCATCCCACGACGGIGGCAAACAGGCTCTTGAGACGGTTCAG |
| | | AGACTTCTCCCAGTTCTCTGTCAAGCCCACGGGCTGACTCCCGATCAAGTIGTAGCGATTGCGTCGCAT |
| | | GACGGAGGGAAACAAGCATTGGAGACTGTCCAACGGCTCCTTCCCGTGTTGTGTCAAGCCCACGGTTTG |
| | | ACGCCTGCACAAGTGGTCGCCATCGCCTCCAATATTGGCGGTAAGCAGGCGCTGGAAACAGTACAGCGC |
| | | CTGCTGCCTGTACTGTGCCAGGATCATGGACTGAC |
| Flexible linker | 10 | GGCCAAGCTGGCCGGGGGCGCCCCCGCCGTGGGCGGGGGCCCCAAGGCCGCCGATAAATTCGCCGCCAC |
| (Example 5) | | C |
| Mutant SPIN | 11 | MTMDRVEKNVKKRKYSEDFLQYGFTSIITAGIEKPQCVICCEVLSAESMKPNKLKRHFDSKHPSFAGKD |
| transposase | | TNYFRSKADGLKKARLDTGGKYHKONVAAIEASYLVALRIARAMKPHTIAEDLLKPAAKDIVRVMIGDE |
| containing | | FVTKLSAISLSNDTVRRRIDDMSADILDQVIQEIKSAPLPIFSIQLDESTDVANCSQLLVYVRYINDGD |
| L124K and | | FKDEFLFCKPLEMTTTARDVFDTVGSFLKEHKISWEKVCGVCTDGAPAMLGCRSGFQRLVLNESPKVIG |
| I509R | | THCMIHRQILATKTLPQELQEVMKSVISSVNFVKASTLNSRLFSQLCNELDAPNNALLFHTEVRWLSRG |
| (substitutions | | KVLKRVFELRDELKTFFNQKARPQFEALFSDKSELQKIAYLVDIFAILNELNLSLQGPNATCLDLSEKI |
| all highlighted; | | RSFQMKLOLWQKKLDENKIYMLPTLSAFFEEHDIEPDKRITMIISVKEHLHMLADEISSYFPNLPDTPF |
| Example 7) | | ALARSPFTVKVEDVPETAQEEFIELRNSDAARTDFSTMPVTKFWIKCLQSYPVLSETVLRLLLPFPTTY |
| | | LCETGFSSLLVIKSKYRSRLVVEDDLRCALAKTAPRISDLVRKKQSQPSH |
| Wild-type | 12 | (accession number: ABF20545) |
| TcBuster | | MMLNWLKSGKLESQSQEQSSCYLENSNCLPPTLDSTDIIGEENKAGTTSRKKRKYDEDYLNFGFTWTGD |
| transposase | | KDEPNGLCVICEQVVNNSSLNPAKLKRHLDTKHPTLKGKSEYFKRKCNELNQKKHTFERYVRDDNKNLL |
| | | KASYLVSLRIAKQGEAYTIAEKLIKPCTKDLTTCVFGEKFASKVDLVPLSDTTISRRIEDMSYFCEAVL |
| | | VNRLKNAKCGFTLQMDESTDVAGLAILLVFVRYIHESSFEEDMLFCKALPTOTTGEEIFNLLNAYFEKH |
| | | SIPWNLCYHICTDGAKAMVGVIKGVIARIKKLVPDIKASHCCLHRHALAVKRIPNALHEVLNDAVKMIN |
| | | FIKSRPLNARVFALLCDDLGSLHKNLLLHTEVRWLSRGKVLTRFWELRDEIRIFFNEREFAGKLNDTSW |
| | | LQNLAYIADIFSYLNEVNLSLOGPNSTIFKVNSRINSIKSKLKLWEECITKNNTECFANLNDFLETSNT |
| | | ALDPNLKSNILEHLNGLKNTFLEYFPPTCNNISWVENPFNECGNVDTLPIKEREQLIDIRTDTTLKSSF |
| | | VPDGIGPFWIKLMDEFPEISKRAVKELMPFVTTYLCEKSFSVYVATKTKYRNRLDAEDDMRLQLTTIHP |
| | | DIDNLCNNKQAQKSH |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A mutant SPIN transposase comprising an amino acid sequence at least 70% identical to full-length SEQ ID NO: 1 and having increased transposition efficiency in comparison to a wild-type SPIN transposase having amino acid sequence SEQ ID NO: 1.

2. A mutant SPIN transposase comprising an amino acid sequence at least 70% identical to full-length SEQ ID NO: 1 and having one or more amino acid substitutions that increase a net charge at a neutral pH in comparison to SEQ ID NO: 1, wherein the mutant SPIN transposase has increased transposition efficiency in comparison to a wild-type SPIN transposase having amino acid sequence SEQ ID NO: 1.

3. The mutant SPIN transposase of claim 2, wherein the one or more amino acid substitutions comprise a substitution with a lysine or an arginine.

4. The mutant SPIN transposase of claim 2 or 3, wherein the one or more amino acid substitutions comprise a substitution of an aspartic acid or a glutamic acid with a neutral amino acid, a lysine or an arginine.

5. The mutant SPIN transposase of any one of claims 1-4, comprising one or more amino acid substitutions from Table 4.

6. A mutant SPIN transposase comprising an amino acid sequence at least 70% identical to full-length SEQ ID NO: 1 and having one more amino acid substitutions in a Specific End Binding Domain; an insertion domain; a Zn-BED domain; or a combination thereof, wherein the mutant SPIN transposase has increased transposition efficiency in comparison to a wild-type SPIN transposase having amino acid sequence SEQ ID NO: 1.

7. A mutant SPIN transposase comprising an amino acid sequence at least 70% identical to full-length SEQ ID NO: 1 and having one or more amino acid substitutions from Table 1.

8. The mutant SPIN transposase of any one of claims 1-7, comprising one or more amino acid substitutions that increase a net charge at a neutral pH within or in proximity to a catalytic domain in comparison to SEQ ID NO: 1.

9. The mutant SPIN transposase of any one of claims 1-8, comprising one or more amino acid substitutions that increase a net charge at a neutral pH in comparison to SEQ ID NO: 1, wherein the one or more amino acids are located in proximity to D185, D251, or E555, when numbered in accordance to SEQ ID NO: 1.

10. The mutant SPIN transposase of claim 8 or 9, wherein the proximity is a distance of about 80, 75, 70, 60, 50, 40, 30, 20, 10, or 5 amino acids.

11. The mutant SPIN transposase of claim 8 or 9, wherein the proximity is a distance of about 70 to 80 amino acids.

12. The mutant SPIN transposase of any one of claims 1-11, wherein the amino acid sequence of the mutant SPIN transposase is at least 80%, at least 90%, at least 95%, at least 98%, or at least 99% identical to full-length SEQ ID NO: 1.

13. The mutant SPIN transposase of any one of claims 1-12, comprising one or more amino acid substitutions from Table 2.

14. The mutant SPIN transposase of any one of claims 1-13, comprising one or more amino acid substitutions from Table 3.

15. The mutant SPIN transposase of any one of claims 1-14, comprising amino acid substitutions I509R, L124K, E219K, and S511N, when numbered in accordance with SEQ ID NO: 1.

16. The mutant SPIN transposase of any one of claims 1-15, comprising amino acid substitutions I509R and L124K, when numbered in accordance with SEQ ID NO: 1.

17. The mutant SPIN transposase of any one of claims 1-16, comprising amino acid substitution I509R, L124K, and E219K, when numbered in accordance with SEQ ID NO: 1.

18. The mutant SPIN transposase of any one of claims 1-17, comprising amino acid substitution I509R and E219K, when numbered in accordance with SEQ ID NO: 1.

19. The mutant SPIN transposase of any one of claims 1-18, comprising amino acid substitution L124K, and E219K, when numbered in accordance with SEQ ID NO: 1.

20. A fusion transposase comprising a SPIN transposase sequence and a DNA sequence specific binding domain, wherein the SPIN transposase sequence has at least 70% identity to full-length SEQ ID NO: 1.

21. The fusion transposase of claim 20, wherein the DNA sequence specific binding domain comprises a TALE domain, zinc finger domain, AAV Rep DNA-binding domain, or any combination thereof.

22. The fusion transposase of claim 20 or 21, wherein the DNA sequence specific binding domain comprises a TALE domain.

23. The fusion transposase of any one of claims 20-22, wherein the SPIN transposase sequence has at least 80%, at least 90%, at least 95%, at least 98/a, or at least 99% identity to full-length SEQ ID NO: 1.

24. The fusion transposase of any one of claims 20-23, wherein the SPIN transposase sequence comprises one or more amino acid substitutions that increase a net charge at a neutral pH in comparison to SEQ ID NO: 1.

25. The fusion transposase of claim 24, wherein the one or more amino acid substitutions comprise a substitution with a lysine or an arginine.

26. The fusion transposase of claim 24 or 25, wherein the one or more amino acid substitutions comprise a substitution of an aspartic acid or a glutamic acid with a neutral amino acid, a lysine or an arginine.

27. The fusion transposase of any one of claims 20-26, wherein the SPIN transposase sequence comprises one or more amino acid substitutions in a Specific end Binding Domain: an insertion domain; a Zn-BED domain; or a combination thereof.

28. The fusion transposase of any one of claims 20-27, wherein the SPIN transposase sequence comprises one or more amino acid substitutions from Table 1.

29. The fusion transposase of any one of claims 20-28, wherein the SPIN transposase sequence has increased transposition efficiency in comparison to a wild-type SPIN transposase having amino acid sequence SEQ ID NO: 1.

30. The fusion transposase of any one of claims 20-29, wherein the SPIN transposase sequence comprises one or more amino acid substitutions that increase a net charge at a neutral pH within or in proximity to a catalytic domain in comparison to SEQ ID NO: 1.

31. The fusion transposase of any one of claims 20-30, wherein the SPIN transposase sequence comprises one or more amino acid substitutions that increase a net charge at a neutral pH in comparison to SEQ ID NO: 1, wherein the one or more amino acid substitutions are located in proximity to D185, D251, or E555, when numbered in accordance to SEQ ID NO: 1.

32. The fusion transposase of claim 30 or 31, wherein the proximity is a distance of about 80, 75, 70, 60, 50, 40, 30, 20, 10, or 5 amino acids.

33. The fusion transposase of claim 30 or 31, wherein the proximity is a distance of about 70 to 80 amino acids.

34. The fusion transposase of any one of claims 20-33, wherein the SPIN transposase sequence comprises one or more amino acid substitutions from Table 2.

35. The fusion transposase of any one of claims 20-34, wherein the SPIN transposase sequence comprises one or more amino acid substitutions from Table 3.

36. The fusion transposase of any one of claims 20-35, wherein the SPIN transposase sequence comprises amino acid substitutions I509R, L124K, E219K, and S511N, when numbered in accordance with SEQ ID NO: 1.

37. The fusion transposase of any one of claims 20-36, wherein the SPIN transposase sequence comprises amino acid substitutions I509R and L124K, when numbered in accordance with SEQ ID NO: 1.

38. The fusion transposase of any one of claims 20-37, wherein the SPIN transposase sequence comprises amino acid substitution I509R, L124K, and E219K, when numbered in accordance with SEQ ID NO: 1.

39. The fusion transposase of any one of claims 20-38, wherein the SPIN transposase sequence comprises amino acid substitution I509R and E219K, when numbered in accordance with SEQ ID NO: 1.

40. The fusion transposase of any one of claims 20-39, wherein the SPIN transposase sequence comprises amino acid substitution L124K, and E219K, when numbered in accordance with SEQ ID NO: 1.

41. The fusion transposase of any one of claims 20-40, wherein the SPIN transposase sequence comprises amino acid substitutions I509R and L124K, when numbered in accordance with SEQ ID NO: 1.

42. The fusion transposase of any one of claims 20-41, wherein the SPIN transposase sequence comprises amino acid substitution S511N, L124K, and E219K, when numbered in accordance with SEQ ID NO: 1.

43. The fusion transposase of any one of claims 20-42, wherein the SPIN transposase sequence comprises amino acid substitution S511N and E219K when numbered in accordance with SEQ ID NO: 1.

44. The fusion transposase of any one of claims 20-43, wherein the SPIN transposase sequence comprises amino acid substitution L124K, and S511N, when numbered in accordance with SEQ ID NO: 1.

45. The fusion transposase of any one of claims 20-44, wherein the SPIN transposase sequence has 100% identity to full-length SEQ ID NO: 1.

46. A polynucleotide that codes for the mutant SPIN transposase of any one of claims 1-19.

47. A polynucleotide that codes for the fusion transposase of any one of claims 20-45.

48. The polynucleotide of claim 46 or 47, wherein the polynucleotide comprises DNA that encodes the mutant SPIN transposase or the fusion transposase.

49. The polynucleotide of any one of claims 46-48, wherein the polynucleotide comprises messenger RNA (mRNA) that encodes the mutant SPIN transposase or the fusion transposase.

50. The polynucleotide of claim 49, wherein the mRNA is chemically modified.

51. The polynucleotide of any one of claims 46-50, wherein the polynucleotide comprises nucleic acid sequence encoding for a transposon recognizable by the mutant SPIN transposase or the fusion transposase.

52. The polynucleotide of any one of claims 46-51, wherein the polynucleotide is present in a DNA vector.

53. The polynucleotide of claim 52, wherein the DNA vector comprises a mini-circle plasmid.

54. A cell producing the mutant SPIN transposase or fusion transposase of any one of claims 1-45.

55. A cell containing the polynucleotide of any one of claims 46-53.

56. A method of genome editing, comprising: introducing into a cell the mutant SPIN transposase of any one of claims 1-19 and a transposon recognizable by the mutant SPIN transposase.

57. A method of genome editing, comprising: introducing into a cell the fusion transposase of any one of claims 20-45 and a transposon recognizable by the fusion transposase.

58. The method of claim 56 or 57, wherein the introducing comprises contacting the cell with a polynucleotide encoding the mutant SPIN transposase or the fusion transposase.

59. The method of claim 58, wherein the polynucleotide comprises DNA that encodes the mutant SPIN transposase or the fusion transposase.

60. The method of claim 58, wherein the polynucleotide comprises messenger RNA (mRNA) that encodes the mutant SPIN transposase or the fusion transposase.

61. The method of claim 60, wherein the mRNA is chemically modified.

62. The method of any one of claims 56-61, wherein the introducing comprises contacting the cell with a DNA vector that contains the transposon.

63. The method of claim 62, wherein the DNA vector comprises a mini-circle plasmid.

64. The method of any one of claims 56-63, wherein the introducing comprises contacting the cell with a plasmid vector that contains both the transposon and the polynucleotide encoding the mutant SPIN transposase or the fusion transposase.

65. The method of any one of claims 56-64, wherein the introducing comprises contacting the cell with the mutant SPIN transposase or the fusion transposase as a purified protein.

66. The method of any one of claims 56-65, wherein the transposon comprises a cargo cassette positioned between two inverted repeats.

67. The method of claim 66, wherein a left inverted repeat of the two inverted repeats comprises a sequence having at least 50%, at least 60%, at least 80%, at least 90%, at least 95%, at least 98%, or at least 99% identity to SEQ ID NO: 3.

68. The method of claim 66, wherein a left inverted repeat of the two inverted repeats comprises SEQ ID NO: 3.

69. The method of any one of claims 66-68, wherein a right inverted repeat of the two inverted repeats comprises a sequence having at least 50%, at least 60%, at least 80%, at least 90%, at least 95%, at least 98%, or at least 99% identity to SEQ ID NO: 4.

70. The method of any one of claims 66-68, wherein a right inverted repeat of the two inverted repeats comprises SEQ ID NO: 4.

71. The method of any one of claims 66-70, wherein the cargo cassette comprises a promoter selected from the group consisting of: CMV, EFS, MND, EF1α, CAGCs, PGK, UBC, U6, H1, and Cumate.

72. The method of any one of claims 66-71, wherein the cargo cassette comprises a CMV promoter.

73. The method of any one of claims 66-72, wherein the cargo cassette is present in a forward direction.

74. The method of any one of claims 66-72, wherein the cargo cassette is present in a reverse direction.

75. The method of any one of claims 56-74, wherein the introducing comprises transfecting the cell with the aid of electroporation, microinjection, calcium phosphate precipitation, cationic polymers, dendrimers, liposome, microprojectile bombardment, fugene, direct sonic loading, cell squeezing, optical transfection, protoplast fusion, impalefection, magnetofection, nucleofection, or any combination thereof.

76. The method of any one of claims 56-75, wherein the introducing comprises electroporating the cell.

77. The method of any one of claims 56-76, wherein the cell is a primary cell isolated from a subject.

78. The method of claim 77, wherein the subject is a human.

79. The method of claim 77 or 78, wherein the subject is a patient with a disease.

80. The method of any one of claims 77-79, wherein the subject has been diagnosed with cancer or tumor.

81. The method of any one of claims 77-80, wherein the cell is isolated from blood of the subject.

82. The method of any one of claims 77-81, wherein the cell comprises a primary immune cell.

83. The method of any one of claims 77-82, wherein the cell comprises a primary leukocyte.

84. The method of any one of claims 77-83, wherein the cell comprises a primary T cell.

85. The method of claim 84, wherein the primary T cell comprises a gamma delta T cell, a helper T cell, a memory T cell, a natural killer T cell, an effector T cell, or any combination thereof.

86. The method of any one of claims 54-85, wherein the primary immune cell comprises a CD3+ cell.

87. The method of any one of claims 56-86, wherein the cell comprises a stem cell.

88. The method of claim 87, wherein the stem cell is selected from the group consisting of: embryonic stem cell, hematopoietic stem cell, epidermal stem cell, epithelial stem cell, bronchoalveolar stem cell, mammary stem cell, mesenchymal stem cell, intestine stem cell, endothelial stem cell, neural stem cell, olfactory adult stem cell, neural crest stem cell, testicular cell, and any combination thereof.

89. The method of claim 87, wherein the stem cell comprises induced pluripotent stem cell.

90. The method of any one of claims 66-89, wherein the cargo cassette comprises a transgene.

91. The method of claim 90, wherein the transgene codes for a protein selected from the group consisting of: a cellular receptor, an immunological checkpoint protein, a cytokine, and any combination thereof.

92. The method of claim 90 or 91, wherein the transgene codes for a cellular receptor selected from the group consisting of: a T cell receptor (TCR), a B cell receptor (BCR), a chimeric antigen receptor (CAR), or any combination thereof.

93. A method of treatment, comprising:
(a) introducing into a cell a transposon and the mutant SPIN transposase or the fusion transposase of any one of claims 1-45, which recognize the transposon, thereby generating a genetically modified cell;
(b) administering the genetically modified cell to a patient in need of the treatment.

94. The method of claim 93, wherein the genetically modified cell comprises a transgene introduced by the transposon.

95. The method of claim 93 or 94, wherein the patient has been diagnosed with cancer or tumor.

96. The method of any one of claims 93-94, wherein the administering comprises transfusing the genetically modified cell into blood vessels of the patient.

97. A system for genome editing, comprising: the mutant SPIN transposase or fusion transposase of any one of claims 1-45, and a transposon recognizable by the mutant SPIN transposase or the fusion transposase.

98. A system for genome editing, comprising: the polynucleotide encoding a mutant SPIN transposase or fusion transposase of any one of claims 1-45, and a transposon recognizable by the mutant SPIN transposase or the fusion transposase.

99. The system of claim 98, wherein the polynucleotide comprises DNA that encodes the mutant SPIN transposase or the fusion transposase.

100. The system of claim 98 or 99, wherein the polynucleotide comprises messenger RNA (mRNA) that encodes the mutant SPIN transposase or the fusion transposase.

101. The system of claim 100, wherein the mRNA is chemically modified.

102. The system of any one of claims 98-101, wherein the transposon is present in a DNA vector.

103. The system of claim 102, wherein the DNA vector comprises a mini-circle plasmid.

104. The system of any one of claims 98-103, wherein the polynucleotide and the transposon are present in a same plasmid.

105. The system of any one of claims 97-104, wherein the transposon comprises a cargo cassette positioned between two inverted repeats.

106. The method of claim 105, wherein a left inverted repeat of the two inverted repeats comprises a sequence having at least 50%, at least 60%, at least 80%, at least 90%, at least 95%, at least 98%, or at least 99% identity to SEQ ID NO: 3.

107. The method of claim 105, wherein a left inverted repeat of the two inverted repeats comprises SEQ ID NO: 3.

108. The method of any one of claims 105-107, wherein a right inverted repeat of the two inverted repeats comprises a sequence having at least 50%, at least 60%, at least 80%, at least 90%, at least 95%, at least 98%, or at least 99% identity to SEQ ID NO: 4.

109. The method of any one of claims 105-107, wherein a right inverted repeat of the two inverted repeats comprises SEQ ID NO: 4.

110. The system of any one of claims 105-109, wherein the cargo cassette comprises a promoter selected from the group consisting of: CMV, EFS, MND, EF1α, CAGCs, PGK, UBC, U6, H1, and Cumate.

111. The system of any one of claims 105-109, wherein the cargo cassette comprises a CMV promoter.

112. The system of any one of claims 105-111, wherein the cargo cassette comprises a transgene.

113. The system of claim 112, wherein the transgene codes for a protein selected from the group consisting of a cellular receptor, an immunological checkpoint protein, a cytokine, and any combination thereof.

114. The system of claim 112 or 113, wherein the transgene codes for a cellular receptor selected from the group consisting of: a T cell receptor (TCR), a B cell receptor (BCR), a chimeric antigen receptor (CAR), or any combination thereof.

115. The system of any one of claims 105-114, wherein the cargo cassette is present in a forward direction.

116. The system of any one of claims 105-115, wherein the cargo cassette is present in a reverse direction.

117. A method of genome editing, which comprises introducing into a cell:
(a) the mutant SPIN transposase of any one of claims 1-19,
(b) a second transposase
(c) a first transposon recognizable by the mutant SPIN transposase but not the second transposase, and
(d) a second transposon recognizable by the second transposase but not the mutant SPIN transposase.

118. The method of claim 117, wherein the second transposase is a hAT transposase.

119. The method of claim 118, wherein the hAT transposase is a TcBuster transposase.

120. The method of claim 119, wherein the TcBuster transposase is a mutant TcBuster transposase comprising an amino acid sequence at least 70% identical to full-length SEQ ID NO: 12 and an amino acid substitution of V377T, E469K, D189A, K573E, E578L, or any combination thereof, when numbered in accordance with SEQ ID NO: 12.
